(19) United States
(12) Patent Application Publication
(10) Pub. No.: US 2024/0350828 A1
(43) Pub. Date: Oct. 24, 2024

(54) Theranostic Methods

(71) Applicant: Board of Regents, The University of Texas System, Austin, TX (US)

(72) Inventors: Forhad Akhter, San Antonio, TX (US); Robert Lyle Hood, San Antonio, TX (US); Yusheng Feng, San Antonio, TX (US); Kathryn Mayer, San Antonio, TX (US); Santiago Manrique Bedoya, San Antonio, TX (US); Chris Moreau, San Antonio, TX (US)

(21) Appl. No.: 18/688,558

(22) PCT Filed: Aug. 31, 2022
(86) PCT No.: PCT/US2022/042274

§ 371 Date: Mar. 1, 2024

Related U.S. Application Data
(60) Provisional application No. 63/239,159, filed on Aug. 31, 2021.

(51) Int. Cl. A61N 5/067; A61K 41/00; A61N 5/06

(52) U.S. Cl. CPC A61N 5/067; A61K 41/0052; A61N 5/0601; A61N 2005/0612; A61N 2005/063

(57) ABSTRACT

A method of treating pancreatic cancer comprising: contacting a pancreatic cancer to be treated with a plasmonic photothermal agent forming a sensitized target; and exposing the sensitized target to light having a resonance wavelength of the plasmonic photothermal agent for a predetermined amount of time; wherein the sensitized target is heated to a sub-hyperthermic temperature that reduces the size of the target.

### PRIORITY PARAGRAPH

This Application is an international application claiming priority to U.S. Provisional Application Ser. No. 63/239,159 filed Aug. 31, 2021, which is incorporated herein by reference in its entirety.

### STATEMENT REGARDING FEDERALLY FUNDED RESEARCH

None.

### BACKGROUND

Pancreatic cancer (PC) has one of the highest mortality rates among all cancers, with a five-year mean survival of around 10%, it is the third most common cause of cancer-related death in the US, and is predicted to be the second by 2030 [1]. The development and progression of this disease is still largely misunderstood as most patients are diagnosed at a late-stage, leaving the effectiveness of standard treatments highly variable [2]. Only 20-30% of patients diagnosed with pancreatic cancer are surgical candidates due to advanced age or proximity of major vasculature [3, 4]. Current treatment methods, such as chemo- and radiotherapy, have relatively poor specificity, systemic side effects, and low efficacy for treatment of pancreatic cancer [5, 6]. The limitations of these techniques and high mortality rate of pancreatic cancer have motivated the development of minimally-invasive treatments.

Among the techniques developed and refined over the past few decades, those using light absorption have received substantial attention. The wavelength-dependent tissue depth reached by a collimated beam is determined by the scattering and absorption of tissue chromophores, and absorbed light generates tissue hyperthermia [7, 8]. As most malignancies often showcase a lower heat tolerance than the surrounding healthy tissue and exposure can be controlled by direction of the light, this approach is considered to have both tissue selective damage and spatial control. Despite the higher localization, the higher power density needed in most laser treatments results in damage to the healthy tissue along the beam path or in nearby tissues [9-11]. In an attempt to overcome this issue, photodynamic therapy (PDT) uses photosensitizers that react with tissue oxygen upon exposure to a specific wavelength [12]. Although the light fluence is reduced, the photosensitizer used in PDT tends to stay in the body for long periods of time, giving the patients a heightened sensitivity to light and can result in complications while undertaking the treatment [9]. An alternative to these treatments is plasmonic photothermal therapy (PPTT). Metallic nanoparticles, when exposed to light at their resonance wavelength, experience a coherent, collective oscillation of the conduction-band electrons that leads to significant light scattering or absorption [9, 13]. This technique typically utilizes light in the near infrared (NIR) range due to the high physiological transmissivity observed at those wavelengths, minimizing undesired tissue absorption and off-target heating [14]. Additionally, PPTT uses metallic nanoparticles, such as gold nanorods (GNRs), as photothermal agents. Due to their optical tunability through geometric manipulation, these GNRs have light absorption up to five times greater than that offered by conventional phototabsorbing dyes [9].

Proper understanding of light-nanoparticle interactions in both healthy and tumorous tissue samples, as well as ex vivo validation of the technique, are required for design of PPTT prior to clinical implementation. A computational model has been developed to study the effects of size and shape in light-nanoparticle interactions [13]. However, the effect on pancreas tissue is yet to be assessed. To properly study the light-nanoparticle effects in pancreas tissue, it is necessary to first understand the thermal and optical properties of the tissue itself. This can be achieved via characterization of these properties of the pancreas. Despite broad knowledge regarding different tissue properties, it is noted that the thermal and optical properties of liver are commonly used for pancreas [15-17]. Furthermore, a limited number of groups have carried out characterization studies for pancreas tissue. Valvano et al. studied the thermal properties of different tissue types, including pancreas, and measured the thermal conductivity of the tissue using a self-heating probe [18]. Review of relevant literature revealed few optical studies of human pancreas tissue, either healthy or diseased. Researchers investigated the human pancreas tissue optical properties for early diagnosis of pancreatic ductal adenocarcinoma (PDAC). Chandra et al. (2007) utilized a fiberoptic probe to measure light absorbance and reflectance by fluorescence and reflectance spectroscopy in visible light range (300-700 nm) [19]. The group used human tissue including healthy pancreas, pancreas tissue suffering pancreatitis, and PDAC in experiments characterizing absorbance in the visible optical range. The optical properties in NIR were not studied in that research. Bashkatov et al. (2011) reported optical properties of human skin, subcutaneous fat, and muscle, but did not explore pancreas tissue [20]. Lee et al. (2013) used optical spectroscopy to diagnose the intraductal papillary mucinous neoplasm (IPMN), one of the precursor lesions of PDAC [21]. More recently, Saccomandi et al. studied the photothermal heating effect of 1064 nm laser illumination on porcine pancreas tissue and measured optical properties of the neuroendocrine human pancreas tumor tissue [15, 16]. A significant item of note is that, for the studies investigating pancreas tissue, all work utilized frozen samples. However, no research has been identified that quantified the difference in optical and thermal properties for fresh versus frozen pancreas tissue samples. Investigation of fresh tissue properties are crucial for the validation and in vivo application of the photothermal therapies. Furthermore, the optical properties of healthy porcine pancreas tissue at 808 and 1064 nm light, as well as a reliable methodology to assess thermal properties of said tissue, have yet to be documented. As these are commonly used laser wavelengths with well documented utility for photothermal therapies, this is a critical gap in the literature that the current research is intended to address [22-24].

Treatments for PC include surgery, radiotherapy, and chemotherapy; however, the 5-year survival rate is <10% (48). This is predominantly due to late-stage symptom presentation which eliminates curative surgical options. The majority of patients rely on non-surgical therapies such as systemic chemotherapy or external beam radiation, neither of which have yielded much improvement in survival over the past few decades. Penetration of systemic chemotherapy into pancreatic tumors is limited by poor local vascular distribution in the pancreas and the fibrotic, desmoplastic barrier that these tumors create, thus leading to poor patient outcomes. New therapeutic approaches which access and target the tumor directly are desperately needed to help improve survival.

### SUMMARY

Photothermal therapies have shown promise for treating pancreatic ductal adenocarcinoma when they can be applied selectively, but off-target heating can frustrate treatment outcomes. Improved strategies leveraging selective binding and localized heating are possible with precision medical approaches such as functionalized gold nanoparticles, but careful control of optical dosage and thermal generation is imperative. However, literature review revealed that many groups assume liver properties for pancreas tissue or rely on insufficiently rigorous characterization studies. These findings motivate a study wherein these properties are measured in healthy samples of fresh and frozen porcine pancreas ex vivo.

Thermal conductivity of the porcine pancreas tissue was measured by utilizing a hot plate and two thermocouples. Optical evaluations assessed light attenuation at the 808 and 1064 nm wavelengths by measuring the light transmittance and reflectance of different tissue thicknesses. In turn, these measurements were input into an inverse adding doubling (IAD) program to estimate the optical absorption and reduced scattering coefficients. Interestingly, pancreas tissue thermal conductivity was demonstrated to have no significant difference between samples that were fresh, frozen for 7 days, or frozen for 14 days. Conversely, optical property assessment exhibited a significant difference between fresh and frozen tissue samples, with increased absorbance and reflectance within the frozen group. However, the optical attenuation values measured were substantially less than that of liver or reported in previous pancreas studies, suggesting wide over-estimation of these properties. These findings are critical to the development of novel therapeutic strategies like PPTT, but perhaps more importantly, are invaluable towards informing better surgical planning and operative technique among the existing thermal approaches for treating pancreas tissue.

Certain embodiments are directed to an intratumoral therapy for precise and local targeting of cancer tissue by utilizing a fiberoptic microneedle device (FMD) for laser and liquid nanoparticle solution (e.g., gold nanoparticle) delivery through a diagnostic technology such as endoscopic ultrasound or a cystoscopy depending on the tumor/cancer tissue location.

The purpose of the research described herein is to investigate the thermal and optical properties of porcine pancreas tissue, including a comparison between fresh and frozen samples. More specifically, the thermal conductivity (k), absorption coefficient (μₐ), reduced scattering coefficient (μₛ′), and attenuation coefficient (μₜ) of healthy pancreatic porcine tissue were characterized when irradiated with both 808 and 1064 nm wavelength laser light. Knowledge of these measurements will aid in the development of analytical models and ex vivo testing that will enable prediction and design of therapeutic approaches at these important wavelengths.

Plasmonic photothermal therapy (PPTT) has potential as a superior treatment method for pancreatic cancer, a disease with high mortality partially attributable to the currently non-selective treatment options. PPTT utilizes gold nanoparticles infused into a targeted tissue volume and exposed to a specific light wavelength to induce selective hyperthermia. Aspects described herein focus on developing this approach within an ex vivo porcine pancreas model via an innovative fiberoptic microneedle device (FMD) for co-delivering light and gold nanoparticles. The effects of laser wavelengths (808 vs. 1064 nm), irradiances (20-50 mW·mm⁻²), and gold nanorod (GNR) concentrations (0.1-3 nM) on tissue temperature profiles were evaluated to assess and control hyperthermic generation. In certain aspects, the FMD is a fabricated silica based light guiding capillary. The optical, fluid, and mechanical characterization of the FMD showed the high light coupling efficiency (˜75%), maximum internal fluid pressure (˜3 MPa), and ability to penetrate a soft tissue without tip buckling. The GNRs had a peak absorbance at ˜800 nm. Results showed that, at 808 nm, photon absorption and subsequent heat generation within tissue without GNRs was 65% less than 1064 nm. The combination of GNRs and 808 nm resulted in a 200% higher temperature rise than the 1064 nm under similar conditions. A computational model was developed to predict the temperature shift and was validated against experimental results with a deviation of <5%. These results show promise for both a predictive model and spatially selective, tunable treatment modality for pancreatic cancer.

Embodiments of the invention are directed to design, fabrication, and characterization of a FMD to determine the platform's mechanical strength, hydraulic resistance, and optical efficiency. The thermal and optical properties of porcine pancreas tissue, including a comparison between fresh and frozen samples, are determined to bridge the identified gap in the literature. In addition, photothermal heating of ex vivo porcine pancreas tissue by NIR light is characterized, both with and without GNRs delivered by the FMD. A computational model is developed and validated utilizing tissue-specific thermal and optical properties to predict the tissue temperature as a function of optical and GNR parameters to provide for a more effective therapy.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Schematic diagram of the experimental setup and the sequential procedure of porcine pancreas tissue thermal conductivity measurement.

FIG. 2. Tissue sample holder with double glass slides and spring loaded screws.

FIG. 3. 808 nm and 1064 nm wavelengths collimated laser beam area measurement through analysis of the reflected beam thermal image.

FIG. 4. Schematic diagram of the setup for (a) light transmittance and (b) reflectance tests.

FIG. 5. Experimental Setup for light transmittance test: (a) at 1064 nm laser; (b) at 808 nm laser; (c) close view of the tissue sample exposed to 808 nm laser, (d) different measurement techniques of light transmittance test.

FIG. 6. Experimental Setup for light reflectance test: (a) reflectance standard measurement, (b) reflectance measurement from tissue and glass slides, (c) different measurement techniques of light reflectance test.

FIG. 7. Thermal conductivity measurements at 40° C. for specimens tested on the same day of excision (Fresh, n=5), specimens frozen for a week (Frozen I, n=6), and specimens frozen for 2 weeks (Frozen II, n=6), and the value reported by Valvano et al. [18].

FIG. 8. Light transmittance (%) vs tissue thickness (mm) graph for both fresh (n=7) and frozen (n=17) porcine pancreas tissue sample. Laser beam intensity: 5 mW·mm⁻² at 808 and 1064 nm wavelengths. Each data point represents mean±1σ (standard deviation) for five consecutive tests with same sample.

FIG. 9. Light reflectance (%) vs tissue thickness (mm) graph for both fresh (n=7) and frozen (n=7) porcine pancreas tissue sample when exposed to 808 and 1064 nm wavelength laser at 5 mW mm⁻² intensity. Each data point represents mean±1σ (standard deviation) of five consecutive tests with same tissue sample.

FIG. 10. Comparison of light attenuation coefficient among IAD results for the fresh and frozen porcine pancreas and results obtained from the literature. (porcine liver and neuroendocrine tumor of human pancreas [15]).

FIG. 11. Effect of thickness on thermal conductivity of porcine pancreas at (A) 40° C. and (B) 50° C.

FIG. 12. (a) FMD-enhanced infusion of GNPs with illumination at 1064 nm (sub-hyperthermia). (b) Plasmonic excitation of GNPs at 808 nm and sub-sequent plasmonic photo-thermal heating (localized hyperthermia).

FIG. 13. (a) Temperature map surrounding 3D array of GNRs under nIR illumination as modeled in COMSOL. (b) SEM image of as-synthesized GNRs.

FIG. 14. CAD model of the FMD.

FIG. 15. EUS biopsy of mass in the head of the pancreas.

FIG. 16. Dual integrating spheres for measuring optical properties of tissue.

FIG. 17. (A) SEM image of the GNRs; Representation of GNRs size-frequency histogram of N=100 distribution as determined from SEM image analysis: (B) GNRs length (95.2±4.7 nm), (C) GNRs width (24.8±1.5 nm), average aspect ratio (length/width): 3.83; (D) UV-vis absorption spectrum of a sample GNR solution (1 nM concentration) with an absorption peak at 813 nm wavelength; Temperature rise (ΔT) vs GNRs concentration graph when exposed to 30 mW·mm⁻² beam at (E) 808 nm, and (F) 1064 nm laser wavelengths. The error bars represent mean±1σ (standard deviation) for n=5 trials. Student's T-test showed the statistical significance between two consecutive GNRs concentrations and control (distilled water) which are represented by ★ symbol. ★=P<0.05, ★★=P<0.01, and ★★★=P<0.001.

FIG. 18. Steady-state temperature of ex vivo porcine pancreas tissue at 3 mm and 6 mm depth from top surface during photothermal heating at different laser irradiations (20, 30, 40, 50 mW·mm⁻²) of both 808 and 1064 nm wavelengths. The error bar represents mean±1σ (standard deviation) for n=5 trials. Statistical analysis (T-test) was conducted between results from 3 mm and 6 mm tissue depths for a fixed laser wavelength and irradiation. The significance of the data is represented by ★ symbol where ★=P<0.05, ★★=P<0.01, ★★★=P<0.001.

FIG. 19. Ex vivo porcine pancreas tissue temperature profile during photothermal heating by FMD only without GNRs at (A) 808 nm, (B) 1064 nm; and with infused GNRs (1 nM, 1 ml) at (C) 808 nm, (D) 1064 nm wavelengths. Three different laser irradiations were tested: 30, 40, and 50 mW mm⁻² with an exposure time of 60 s followed by free convection cooling. Error bars represent mean±1σ (standard deviation) for n=5 trials. Statistical analysis (Student's T-test) between the maximum tissue temperatures of each set of two consecutive laser irradiations for a given wavelength showed the significance of the data (P<0.01).

FIG. 20. Ex vivo porcine pancreas tissue temperature vs GNR concentration graphs for three different laser irradiations (30, 40, and 50 mW·mm⁻²) at (A) 808 nm and (B) 1064 nm wavelengths. Laser exposure time was fixed at 60 s. Error bars represent mean±1σ (standard deviation) for n=5 trials. Statistical analysis (T-test) between tissue temperatures from different irradiation for a fixed wavelength and GNR concentration showed the significance of the data (P<0.01).

FIG. 21A-21F. (A) Simulation results after 20 minutes of tissue photothermal heating by an 808 nm collimated beam with irradiance of 40 mW·mm⁻²; Comparison between simulation and experimental results for collimated beams at (B) 808 nm, 40 mW·mm⁻² irradiance, 3 mm tissue depth; (C) 1064 nm, 20 mW·mm⁻² irradiance at 6 mm tissue depth; (D) simulation results after 1 minute of tissue photothermal heating by FMD at 1064 nm, 40 mW·mm⁻² irradiance; Comparison of simulation and experimental results for FMD at (E) 808 nm, 40 mW·mm⁻²; (F) 1064 nm, 40 mW·mm⁻² irradiances. Error bars represent mean±1σ (standard deviation) for n=5 trials.

FIG. 22. Relation between GNRs absorbance and concentration. The characteristic equation of the trend line was utilized to evaluate the GNRs concentration (0.1-3 nM) from the absorbance result from UV-Vis spectrometer.

FIG. 23. Findings of the preliminary tests to set the range of laser irradiation for ex vivo porcine pancreas tissue photothermal heating by 808 and 1064 nm laser.

FIG. 24. Ex vivo porcine pancreas tissue photothermal heating and free convective cooling graphs at (A) 808 nm and (B) 1064 nm wavelengths of the collimated laser beam. Four different laser irradiations were tested (20, 30, 40, 50 mW·mm⁻²) for measuring tissue temperatures at 3 and 6 mm depths. Graphs show the mean values of n=5 trials.

FIG. 25. Ex vivo porcine pancreas tissue photothermal heating by FMD utilizing both laser (808 nm; 40 mW·mm⁻²), and GNRs solution (1 nM, 1 ml) and the temperature measurement by capturing image through the thermal camera.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Located in the retroperitoneum, the pancreas is not easily accessed by other minimally invasive techniques such as computed tomography (CT) or magnetic resonance image-guided therapies. Endoscopic ultrasound (EUS), developed in the mid-1980s, has become the gold-standard for diagnosing PC due to its ability to provide high resolution imaging and a means to access the lesion for biopsy or therapeutics (5). EUS-guided direct injection of chemotherapy and brachytherapy agents have shown some mild promising results in patient outcomes (49-52). EUS-guided thermal ablation achieved by passage of a radiofrequency catheter directly into the tumor (radiofrequency ablation; RFA) has been shown to be feasible, although with significant rates of pancreatitis (53). EUS-guided laser ablation (laser interstitial thermal therapy; LITT) in normal pancreas has recently been successfully demonstrated in a pig model and a pilot clinical trial, but control of the zone of ablation is highly dependent on tissue properties (54, 55). Variations in pancreas homogeneity suggest a wide range of values, but the distribution has not been explored in the literature. Without tissue selective, tumor-directed therapies, the risk of normal tissue damage and subsequent complications such as pancreatitis remains high.

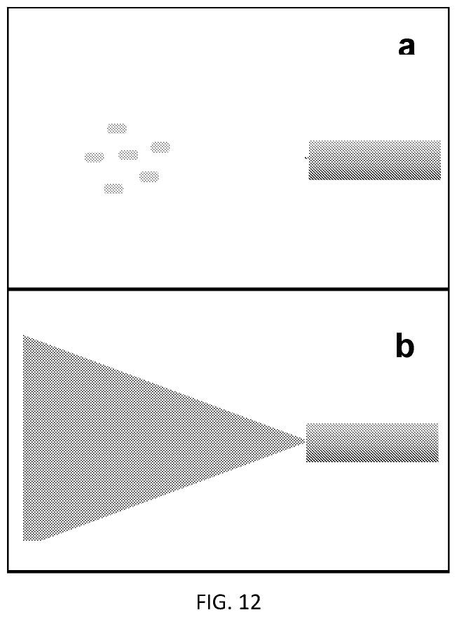

A promising emerging technology for treatment of unresectable PC is plasmonic photothermal therapy (PPTT) (56). This mode of therapy utilizes laser light and gold nanoparticles (GNPs) as heating agents to induce hyperthermia and ablate tumorous tissue. GNPs can be tuned so that they absorb near infrared light (˜800-1000 nm). This spectral region has a high physiological transmittivity (14), thus allowing maximum GNP heating while minimizing undesired tissue effects. The use of functionalized GNPs allows treatment to be targeted to a tumor region and restricts the zone of thermal damage to a highly localized volume, sparing normal tissue. GNPs also show excellent ultrasound image contrast. By combining the targeted intratumoral therapy of PPTT with the diagnostic imaging technology of endoscopic ultrasound, an image-guided theranostic approach is designed which precisely and locally targets PC. Elements for the design include: (1) The spatial distribution of GNPs in pancreatic tumor tissue must be well understood, and (2) The ability to accurately tailor and predict the volume of ablation in PPTT of pancreatic tumor tissue must be developed. The spatial distribution of GNPs in pancreatic tumor tissue needs to be characterized and controlled. PPTT-induced thermal ablation volumes need to be predicted and validated in pancreatic tumor tissue. It is contemplated that GNP delivery via fiber optic microneedle device (FMD) accompanied with heating to sub-hyperthermic temperatures (˜40° C.) via IR laser illumination at 1064 nm over a clinically realistic (˜10 min) duration will statistically significantly improve the volume of dispersal as compared to delivery without laser heating. It is further contemplated that antibody targeting will significantly improve the percentage of GNPs localized inside the tumor as compared with polymer-coated GNPs. It is also contemplated that the volume of the thermal ablation zone resulting from PPTT will match the volume infused with GNPs as described by ultrasound imaging, to within the resolution limitations of the US. FIG. 12 shows a schematic of the proposed approach, including gentle FMD-delivered heating via illumination at 1064 nm to enhance GNP infusion, followed by GNP-mediated localized photothermal heating upon illumination at 808 nm.

Described herein is a theranostic approach to treating PC. The methods use advanced ultrasound imaging along with photothermal heating of gold nanoparticles to treat PC in a local, targeted manner while sparing healthy tissue and reducing side effects such as pancreatitis in comparison with other methods. The combination of EUS image-guided delivery with a therapeutic modality which targets tumorous tissue with superior localization differentiates the current methods from the existing techniques (RFA, LITT, etc.) described in the above section.

Beneficial aspects of the invention include one or more of localization of functionalized GNPs, both targeted and non-targeted, in pancreatic tumor; infusing GNP in pancreatic tissue and precisely delivering GNPs to a localized region using endoscopically-employable methods; computational modeling of the optical properties of healthy and tumorous pancreas tissue to predict the zone of thermal ablation resulting from a given GNP distribution using various laser energy settings; computational modeling of the zone of thermal ablation associated with PPTT in pancreas; computational modeling of both temperature field and damage zone of ablation to provide a basis for outcome prediction and treatment planning.

The compositions and/or methods described herein enable the development of a targeted and localized intratumoral theranostic modality utilizing GNP-mediated photothermal heating. In addition, the fundamental data obtained on normal and tumorous pancreatic tissue properties will be widely applicable in broad areas from diagnostic imaging to therapy. Also, the comparison of ex vivo tissue results in both porcine and human pancreas can serve to evaluate the widely-used porcine model's validity.

### I. PLASMONIC PHOTOTHERMAL THERAPY

In some embodiments, a gold nanomaterial can be at least one of a gold nanorod and a gold nanosphere (AuNS). In some embodiments, the gold nanomaterial can be a gold nanorod (AuNR). Gold nanorods can be utilized in some embodiments where the irradiation source includes a particular emission wavelength or wavelength range that can be absorbed by nanorods.

In some embodiments, the gold nanomaterial can be a gold nanorod having a length dimension of from about 15 nm to about 50 nm, from about 20 nm to about 50 nm, from about 20 nm to about 40 nm, from about 20 nm to about 35 nm, from about 20 nm to about 30 nm, from about 22 nm to about 30 nm, or from 22 nm to about 28 nm. In some embodiments, the gold nanorod can have a length dimension of about 26 nm. In some embodiments, the gold nanorod can have a length dimension of about 25 (±3) nm.

In some embodiments, the gold nanomaterial can be a gold nanorod having a width dimension of from about 1 nm to about 15 nm, from about 2 nm to about 10 nm, from about 5 nm to about 15 nm, from about 5 nm to about 10 nm, or from about 5 nm to about 7 nm. In some embodiments, the gold nanorod can have a width dimension of about 5 nm. In some embodiments, the gold nanorod can have a width dimension of about 5 (±0.5) nm. In some embodiments, the gold nanorod can have a width dimension of about 6 (±1) nm.

In some embodiments, the gold nanomaterial can be a gold nanorod having an aspect ratio of from about 2 to about 10, from about 3 to about 10, from about 3 to about 8, from about 4 to about 7, from about 4 to about 10, from about 3 to about 5, from about 2 to about 6, or from about 3 to about 6. In some embodiments, the gold nanorod can have an aspect ratio of about 4.2.

In some embodiments, the gold nanomaterial can absorb wavelengths of light in the near-infrared (NIR) spectrum. In some embodiments, the gold nanomaterial can absorb wavelengths of light between about 750 nm and about 1250 nm. In some embodiments, the gold nanomaterial is a gold material that can have a maximum absorption peak of about 800 nm (in other words, the nanomaterial can have a UV-vis maximum absorption peak of about 800 nm). In some embodiments, the gold nanomaterial can be a gold nanorod that can absorb wavelengths of light in the NIR spectrum, e.g., from about 750 nm to about 1250 nm, with an absorption maximum of about 800 nm.

In some embodiments, the method can comprise targeting the gold nanomaterial to at least one of an integrin of the cancer cell and a cell nuclear membrane of the cancer cell. In some embodiments, the gold nanomaterial can be conjugated to a targeting moiety that can be configured to specifically target particular areas of the cell, including, but not limited to, surface integrins, cell nuclei, etc. In some embodiments, the gold nanomaterial can be conjugated to one or more Arg-Gly-Asp (RGD) peptides. RGD peptides can specifically bind to a wide number of surface integrins, including but not limited to αvβ3, α3β1, and α5β1 integrins. In some embodiments, the gold nanomaterial can be conjugated to one or more Nuclear Localization Signals (NLS). A NLS is an amino acid sequence that ‘tags’ a protein for introduction into the cell nucleus. In some embodiments, the gold nanomaterial can be conjugated to one or more Bovine Serum Albumin (BSA) moieties. In some embodiments, the gold nanomaterial can be conjugated to one or more Rifampicin (RF) moieties. BSA and RF-conjugated gold nanomaterials can enhance the rate of endocytosis of gold nanomaterials and hence their concentration inside the cancer cell.

In some embodiments, the gold nanomaterial can be conjugated to a moiety (i.e., ligand) that can increase the biocompatibility of the gold nanomaterial. In some embodiments, the gold nanomaterial can be conjugated to one or more Poly-Ethylene Glycol (PEG) moieties. PEG is a polyether compound that can increase the biocompatibility of the gold nanomaterial.

In some embodiments, the gold nanomaterial can be conjugated to only one type of moiety. In some embodiments, the gold nanomaterial can be conjugated to more than one type of moiety, for example, the gold nanoparticle can be conjugated to a targeting moiety and a moiety that increases biocompatibility of the gold nanomaterial. In some embodiments, a single particle of gold nanomaterial (e.g., a single nanorod or nanosphere) can be conjugated to one or more types of moieties. In some embodiments, each particle of gold nanomaterial can be conjugated to a single type of moiety.

In some embodiments, the irradiation source can comprise a single emission wavelength or a range of emission wavelengths. In some embodiments, the emission wavelength range can be a wavelength range that causes minimal or no cellular damage. In some embodiments, the emission wavelength range can be in the near-infrared wavelength range, e.g., from about 750 nm to about 1250 nm. In some embodiments, the irradiation source can comprise a single emission wavelength from about 750 nm to about 1250 nm. In some embodiments, the irradiation source can be a laser with a single emission wavelength of from about 750 nm to about 1250 nm. In some embodiments, the irradiation source can be a laser with an emission wavelength range of from about 750 nm to about 1250 nm. In some embodiments, the irradiation source can be an 808 nm diode laser.

### II. FIBEROPTIC MICRONEEDLE

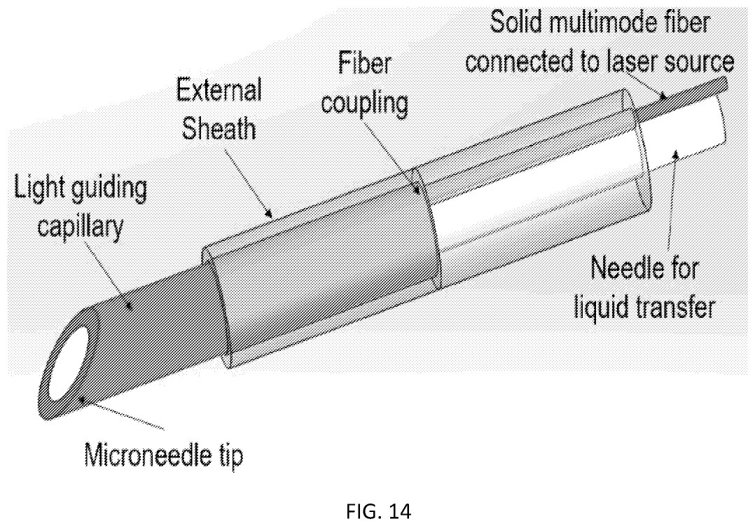

Certain embodiments employ a fiber optic microneedle device. The fiber optic microneedle device (FMD) is a microneedle catheter capable of penetrating soft tissues and co-delivering laser light and fluid agents (see FIG. 14). The FMD was adapted to enhance the volumetric dispersal of macromolecules delivered to the brain through convection-enhanced delivery (CED) by concurrent delivery of sub-lethal photothermal hyperthermia.

Embodiments of the invention provide a non-metal needle comprising structure for transmitting light, which is capable of piercing human tissue, and has a maximum diameter in the range of about 100-300 micron. Also included are needles comprising a base having an outer diameter in the range of about 100-300 micron and a tip having an outer diameter in the range of about 5-50 micron. Further provided are needles comprising a base having an outer diameter in the range of about 100-200 micron and a tip having an outer diameter in the range of about 5-40 micron. Other embodiments provide needles comprising a base having an outer diameter in the range of about 100-150 micron and a tip having an outer diameter in the range of about 5-20 micron. Certain embodiments provide needles comprising a base having an outer diameter in the range of about 100-125 micron and a tip having an outer diameter in the range of about 5-10 micron.

A non-metal material or “non-metal” as used in this disclosure refers to any material that is a poor conductor of heat and electricity. Non-metals in accordance with the present invention can also include materials having a thermal conductivity (at about 25° C.) of about 5 k (W/mK) or less, such as about 2-4 k, or such as about 1 k or less. Silica or silica-based materials or fibers, even though they may contain metals in their compositions are non-metals according to the invention. Ceramics, quartz, plastics, and polymers are also non-metals according to the invention, including many other materials having similar properties. In contrast, aluminum, copper, iron, alloys, brass, nickel, silver, gold, lead, molybdenum, zinc, magnesium, stainless steel, etc. for example are examples of metals.

Certain embodiments provide needles of comprising a hollow core having an inner diameter in the range of about 1-8 micron. Other embodiments provide a needle having a length of about 0.5-6 mm. Embodiments of the present invention provide the needle having a length of about 1-3 mm. Some embodiments provide a needle comprising a hollow core having an inner diameter in the range of about 1-5 micron.

Certain embodiments provide a needle wherein the light-transmitting material is silica. Other embodiments provide a needle comprising multi-mode silica fiber or single-mode silica fiber, and any combination thereof. Embodiments of the present invention provide a needle comprising a flat or non-tapered tip, a tapered tip end, wherein the needle has a first taper defined by an outer diameter that becomes increasingly smaller along a length of the needle toward the tip end and a second taper defined by an outer diameter that becomes increasingly smaller within 10-20% of the tip end based on overall needle length, and any combination thereof.

Certain embodiments of the present invention provide a needle comprising a light-blocking coating. In some embodiments the needle structure is formed from heating and stretching a silica-based fiber cylinder or rod, having a first average outer diameter along the length of the fiber, until a second outer diameter smaller than the first is obtained in a region of the fiber and breaking the fiber at a point in the second smaller diameter region. In embodiments, due to variations in diameter along the length of the fiber, it may be appropriate to refer to the needles as having a minimum outer diameter of a selected dimension. In some embodiments, the breaking of the fiber involves stopping the heating and stretching of the fiber, cooling the fiber, and mechanically breaking the fiber in the needle. Other embodiments provide a needle, wherein breaking of the fiber involves direct laser heating at a point in the second smaller diameter region combined with stretching of the fiber at a rate sufficient to obtain a third outer diameter smaller than the second and sufficient to break the fiber at a point in the third smaller diameter region to form a tapered tip.

The methods as described herein can use a fiberoptic microneedle device comprising: (a) one or more microneedles; (b) a support member to which the needles are secured; and (c) a ferrule comprising one or more holes for each of the needles, wherein the ferrule is operably configured to provide mechanical support to each needle at all or some portion of the length of the needle. Further included in some embodiments is a fiberoptic microneedle device comprising: (a) one or more silica-based needles capable of guiding light and comprising a length of about 0.5-6 mm, a base having an outer diameter in the range of about 100-150 micron, and a tip having an outer diameter in the range of about 5-20 micron; (b) a support member to which the needles are secured; and (c) a ferrule comprising one or more holes for each of the needles, wherein the ferrule is operably configured to provide mechanical support to each needle at all or some portion of the length of the needle. In certain aspects the microneedle is configured to deliver plasmonic phototherapeutic agents to a location, such as a tumor site (e.g., a pancreatic tumor or the like).

In certain aspects a robust and efficient FMD can be used to penetrate pancreas tissue to induce hyperthermia in a short time (ΔT˜5° C. is 60 s) through infusing controlled light intensity (10-50 mW/mm²) and GNRs concentrations (0.1-3 nM). Selective tissue heating would allow the GNRs to absorb and dissipate heat without affecting the tissue due to the high transmissivity and low absorbance of the tissue at the 800 nm range. This method can be beneficial in reducing unwanted tissue damage to the surrounding healthy tissue during the thermal ablation process.

In one example, a smaller multimode fiber was fusion spliced with the annular core of a light guiding capillary to achieve higher light coupling efficiency. The size of capillary and multimode fiber was selected to keep the size of FMD similar to a standard 28G needle while ensuring a high-quality fusion splicing joint between both fiber cores. As the commercial fusion splicers are made to handle similar diameter fibers, it was necessary to optimize different fusion splicing parameters by trial and error. The fusion splicing loss was first estimated by the theoretical equations from literature review. Then, the theoretical values were compared against experimental values obtained from light transmission measurements.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Characterization of Thermal and Optical Properties in Porcine Pancreas Tissue

### A. Methods

Tissue Sample Collection. Tissue samples were obtained from an USDA approved meat processing facility. The samples were excised soon after the animals were sacrificed (<10 minutes). Samples were placed in individual bags and stored inside a chilled container to maintain refrigeration and slow degradation during transport. Freezing was avoided to prevent formation of ice crystals inside the tissue that could rupture cell membranes and ultimately modify tissue properties. Upon arrival to the laboratory, the tissue samples were washed in phosphate-buffered saline (PBS) solution. Sample preparation varied depending on the experiment: thermal conductivity experiments required specimens with a relatively larger cross-sectional area (˜25 cm²) and tissue thickness (0.9-1.4 cm), whereas the light transmissivity experiments required specimens with a smaller cross-sectional area (˜9 cm²) and tissue thickness (0.2-1 cm). The specimens for both experiments were sliced from the fresh samples using a scalpel and the dimensions estimated using calipers and image processing software (ImageJ) [25]. ImageJ can estimate the dimensions of an unknown object through comparison of a second object with a known if both are placed in a single plane perpendicular to the camera. Samples considered “fresh” were experimentally assessed within 3 hours of collection, while “frozen” samples were stored individually in a −80° C. freezer.

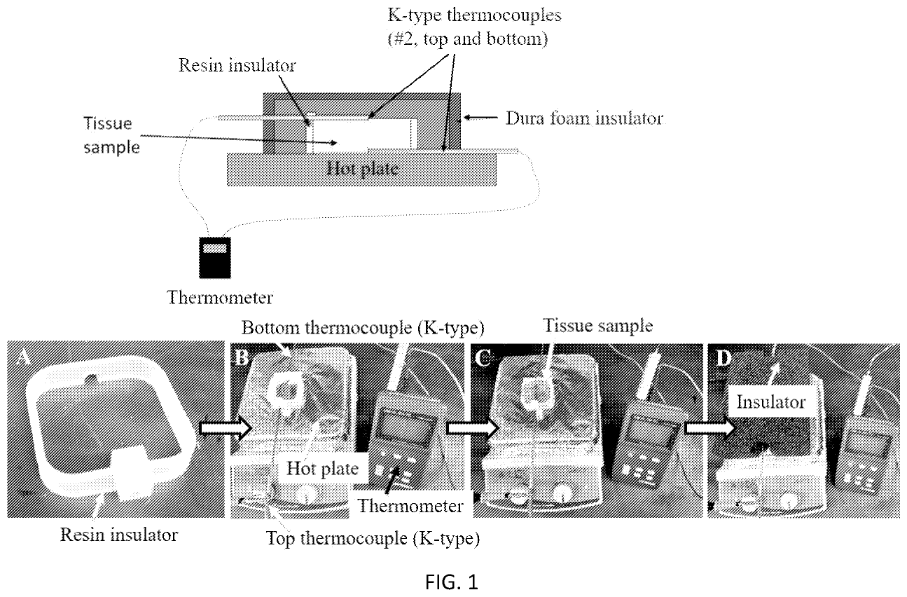

Thermal Conductivity Measurement. Thermal conductivity measurements were carried out using a hot plate (Fisher Scientific; Hampton, NH) covered with aluminum foil. An Omega thermometer (OM-HL-EH-TC, Omega Engineering, Norwalk, CT) and two K-Type thermocouples were used to measure temperature (Fluke Co; Everett, WA). Both thermocouples were calibrated against a cold junction prior to conducting the experiments. The accuracy of the temperature measurements was +/−0.05% and the resolution was 0.1° C. Additionally, a glass thermometer was used to measure room temperature, and a 3D printed border/fixture (high temperature resin, low thermal conductivity at 0.15 Wm⁻¹ K⁻¹ and thermal expansion at 79.6 μm·m⁻¹ C⁻¹) was used to minimize the convective heating coming from the sides, as well as to hold both thermocouples at a fixed distance of 1 cm. Lastly, an external dura foam insulating cover was placed over the hot plate to minimize the convective effects of ambient air. A schematic of the experimental setup and the apparatus utilized are shown in FIG. 1.

The thermal conductivity of tissue was measured based on the mathematical formulation for conservation of energy across a wall or a sample subject to a set temperature in one side and convection on the other side, as shown in Equation 1 [27]:

$$K Δ {T}_{1} = Lh Δ {T}_{2} \qquad ( 1 )$$

where k is the thermal conductivity of the medium, ΔT₁ is the temperature gradient between the two surfaces, L is the thickness of the sample, h is the heat transfer coefficient of free flow of air, and ΔT₂ is the temperature difference between the outermost surface and the ambient. The measurements taken were those obtained when steady state was reached (30-40 minutes, temperature change of each thermocouple <0.1° C. for 10 min interval). Three different types of materials were tested: polydimethylsiloxane (PDMS) (Sylgard™ 184 elastomer) [28], chicken breast [29], and porcine pancreas tissue. Tissue samples were sliced as per the size of the fixture (resin insulator). Each individual tissue sample underwent only one test cycle to avoid errors in the values associated to the change in properties of the tissue due to prolonged exposure to heat. In one experiment, the known properties of a PDMS block and chicken specimens were independently utilized for testing and validation of the experimental setup.

Light Transmission Test in Pancreas Tissue. Previous work described in Manrique et al. presented computational models which required input of porcine pancreas tissue optical properties such as absorption coefficient, scattering coefficient, and attenuation coefficient at a specific wavelength [13]. Numerous approaches to investigate tissue optical properties in both ex and in vivo environments have been described in the literature. Some researchers have utilized numerical simulations to predict tissue specific optical properties. For example, Patterson et al. (1988) introduced Monte Carlo simulation to evaluate reflectance and transmittance of biological tissue, and compared the results against in vivo studies [30]. Prahl and Jacques reported experimental designs using both single or double integrating sphere (photodetector) setups for measuring transmittance and reflectance of ex vivo tissues [31-33]. They also developed the inverse adding doubling (IAD) method, a numerical approach for approximating the optical properties based on experimental measurements from the integrating sphere setups [34]. The current study closely followed experimental procedures reported by Prahl et al. using a single integrating sphere to evaluate the light transmittance and reflectance through porcine pancreas tissue samples. IAD was utilized to approximate the absorption and scattering coefficients.

Light attenuation coefficient (Ucfr) quantifies how easily photons can penetrate a tissue layer, as well as the average mean free path of photons traveling through the tissue, with a higher attenuation coefficient being inversely proportional with light transmittance [34]. It is a unique property of the specific tissue sample which varies depending on the tissue physiological properties. This property is independent of tissue thickness: i.e., the mean free path of photon propagation through the tissue layer is constant. Higher attenuation coefficient represents quick decrease of photon energy when travelling through the tissue layer. Theoretically, attenuation coefficient is a combination of light absorption coefficient (μₐ), scattering coefficient (μₛ), and anisotropy (g) (equation 2, 3). According to the light diffusion approximation [32]:

$${μ}_{eff} = \sqrt{3 {μ}_{a} ( {{μ}_{s}}^{′} + {μ}_{a} )} \qquad ( 2 )$$

$${{μ}_{s}}^{′} = {μ}_{s} ( 1 - g ) \qquad ( 3 )$$

Here, μₛ′ is the reduced scattering coefficient which depends on the anisotropy factor (g). When a photon interacts with a tissue layer, it can be deflected by a certain angle from the original path. Anisotropy (g) is the cosine of that deflection angle and ranges from 0 to 1. Both μₐ and μₛ′ can be approximated through IAD given that the transmittance and reflectance of the tissue sample are known from experimental data.

Light transmittance (M_{T}) is defined as the ratio of the transmitted light intensity, T, and the incident light intensity, T₀ (Equation 4).

$${M}_{T} = \frac{T}{{T}_{0}} \qquad ( 4 )$$

Light reflectance (M_{R}) is defined as the ratio of the reflected light intensity from tissue surface, R, and the maximum reflected light intensity from a reflectance standard, R_{standard} (Equation 5) [32]. This study utilized reflectance standard which can reflect 99% of the incident light within the range of 250-2500 nm wavelengths (Spectralon® Diffuse Reflectance Standards, Labsphere Inc., North Sutton, NH).

$${M}_{R} = \frac{R}{{R}_{s t andard}} \qquad ( 5 )$$

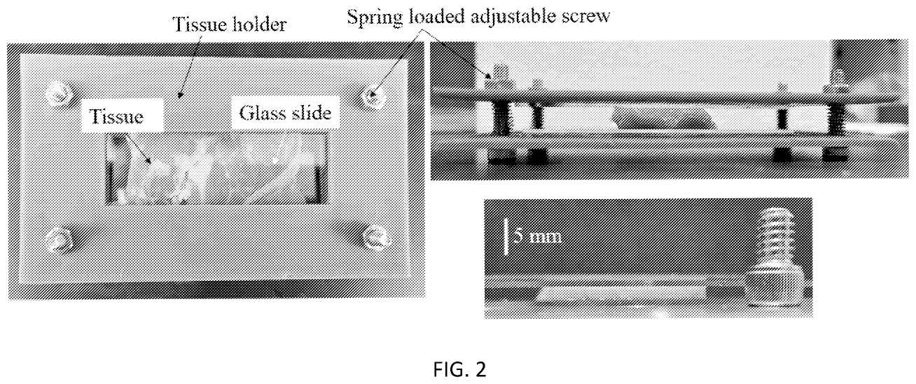

For the experiments, PBS rinsed fresh tissue samples were cut to the desired thickness with a scalpel. The layer thickness of the thin sliced tissue samples was in the range of 1-10 mm. Prahl et al. recommended a method of placing the tissue sample between two glass slides to flatten the irregular tissue surface and ensure more uniform specular reflection. To follow this method, tissue samples were placed on a glass slide (Plain glass slide, Thermo Fisher Scientific, Waltham, MA) attached to a 3D printed holder (Grey resin material, RS-F2-GPGR-04, Formlabs, Somerville, MA) (FIG. 2). Another glass slide along with, the holder, was placed on top of the tissue sample. While placing the glass sides, a small amount of PBS was used to soak the tissue surfaces, reducing the likelihood of air bubble formation. The force between these two glass slide holders was adjusted by four spring loaded screws as shown in FIG. 2.

All the screws were tightened such that the glass slides are in full contact with the top and bottom surfaces of the tissue, ensuring a flat surface with no air gap or uneven deformation of the tissue layer. Once the tissue sample was placed between the two glass slides, the thickness of the tissue layer was measured by taking an image from the side (FIG. 2) and using ImageJ software for post analysis. An average of five readings from ImageJ was calculated for each individual specimen to estimate the tissue layer thickness. Two images were captured before and after placing the tissue sample between the glass slides to compare the deformation of the layer due to compression [35].

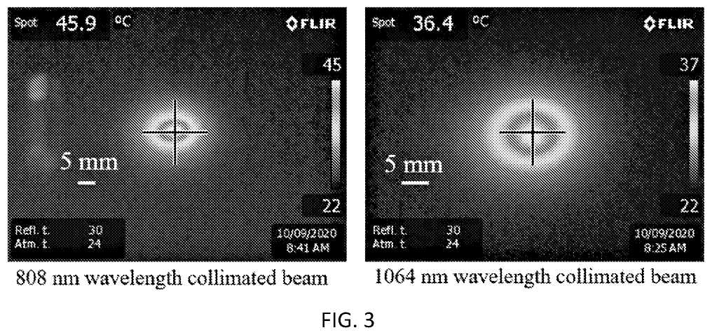

The optical study utilized two different continuous wave laser sources with collimated beam outputs at 808 nm (LRD-0808-PFR-01000-03, Laserglow Technologies) and 1064 nm (YLR 10-1064-LP, IPG photonics) wavelengths. The collimated beam areas for both laser sources were estimated by taking a thermal image (E40, FLIR thermal camera) of the beam reflection and post processing the image using ImageJ (FIG. 3, edges of central white color). The area was measured at 24 and 19.6 mm² for the 808 and 1064 nm beams, respectively.

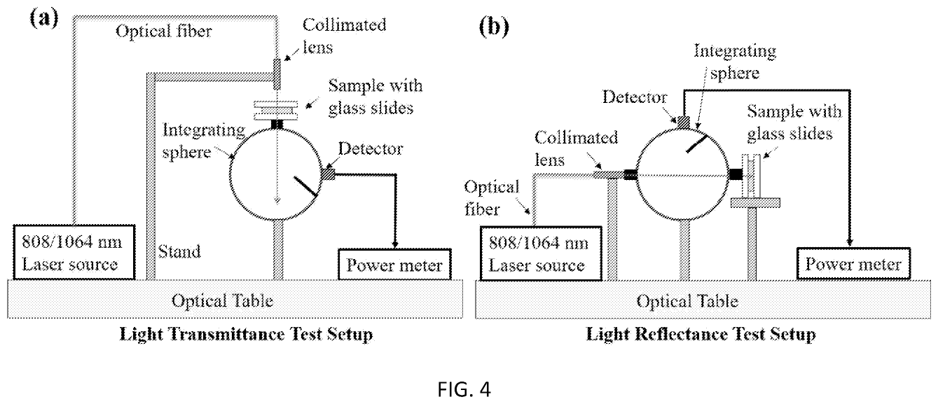

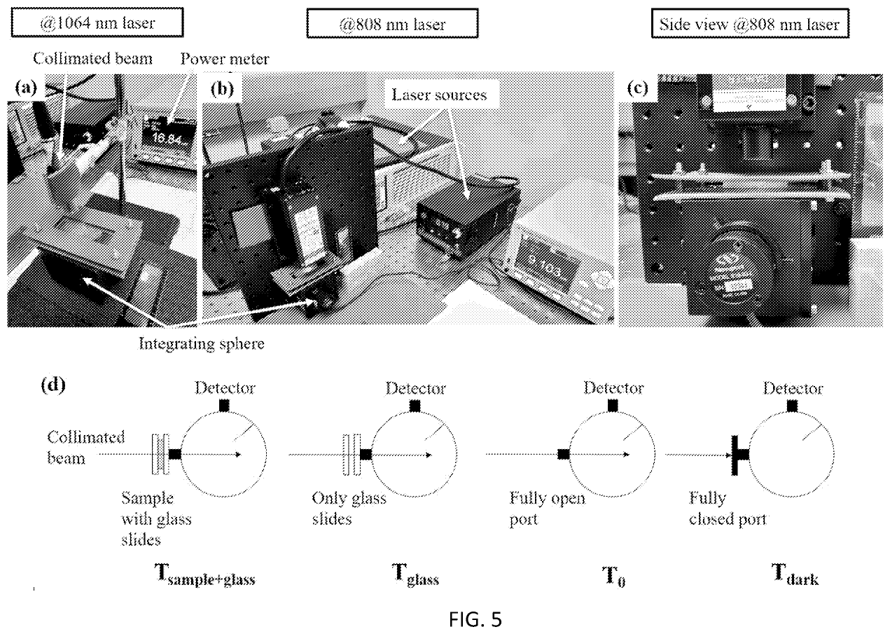

These areas were used to measure the beam intensity which was kept constant at 5 mW·mm⁻² for both laser sources to avoid photothermal damage of the tissue. A preliminary test showed that this low laser irradiation would not increase tissue temperature more than 1° C. The experimental setup included a laser source with collimated beam output, an integrating sphere for detecting the transmitted and reflected light, and a power meter for measuring the light power (FIG. 4). For light transmittance tests, the tissue samples were placed vertically in between the laser source and the integrating sphere (FIG. 4A and FIG. 5). The tissue holder was placed directly atop the integrating sphere and arranged with a fixed distance (12 mm) from the collimated beam source. The transmitted beam through the tissue sample entered the sphere without any obstruction as the open port area of the integrating sphere (126.7 mm²) was far larger than the collimated beam area for both laser sources. The amount of light transmitted (T_{sample+glass}) through the glass slides and tissue sample was measured in real time by the power meter.

A set of control experiments were conducted (FIG. 5d). First, light transmittance through the glass slides with no tissue sample was measured (T_{glass}). During this test, the gap between the glass slides was kept similar to the tissue layer thickness. Two other calibration experiments included transmittance without any sample and glass (T₀), and transmittance when the port of the integrating sphere is fully blocked, i.e., no light condition (T_{dark}). Equation 6 was used to obtain the actual light transmittance (M_{T}) through the tissue sample by taking the control experimental results into account.

$${M}_{T} ( \% ) = ( \frac{{T}_{sample + glass} - {T}_{glass} - {T}_{dark}}{{T}_{0} - {T}_{dark}} ) * 100 \qquad ( 6 )$$

Similar procedures were followed while conducting experiments wherein the tissue sample and layer thickness were varied. In this test, both fresh and frozen samples (stored in the freezer for <3 weeks) were utilized for a comparison study. A total of 7 fresh samples and 17 frozen samples were used to measure the light transmittance. Each experiment was repeated three times to get an average light transmittance for each tissue layer.

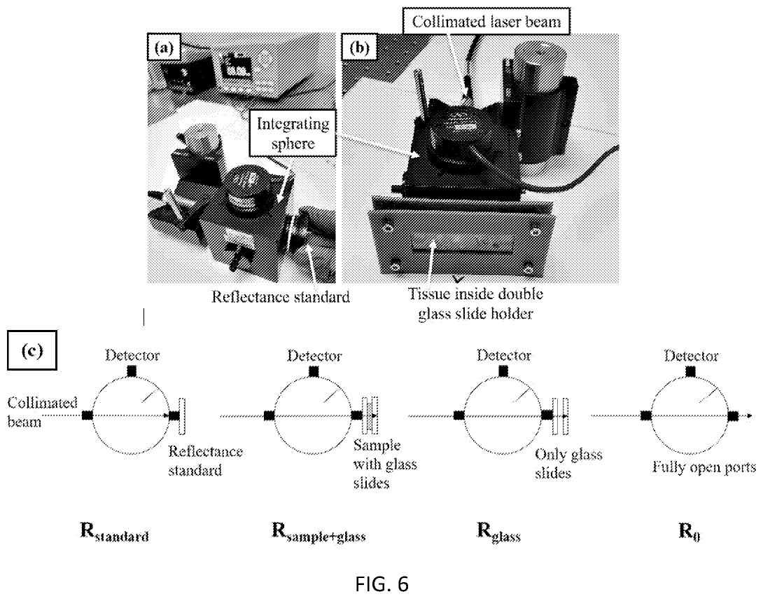

FIG. 4B and FIG. 6 show the experimental setup for the light reflectance test where the integrating sphere was placed horizontally in between the collimated beam and tissue sample. The integrating sphere used in this test was similar to the transmittance test, except there were two open ports in line with the beam path. The collimated lens was in line with one open port of the sphere while the tissue sample, along with the glass slides, was placed in contact with the distal port of the sphere. Both the lenses and glass slides (containing the tissue sample) were in contact with the open ports of the integrating sphere. As the open port area of the sphere had a far larger diameter than the beam area for both laser wavelengths, the collimated beam from the laser source travelled through the sphere without reflection. The photodetector of the sphere only captured the reflected beam from the tissue, glass surface, and glass-tissue interfaces. This reflected light (R_{sample+glass}) was measured by the power meter. Tissue sample thickness varied within the range of 2-8 mm. A set of control experiments were conducted that includes the light reflectance measurement from a reflectance standard (R_{standard}), the glass slides only (R_{glass}), and with both ports of the integrating sphere in open condition (R₀) (FIG. 6c).

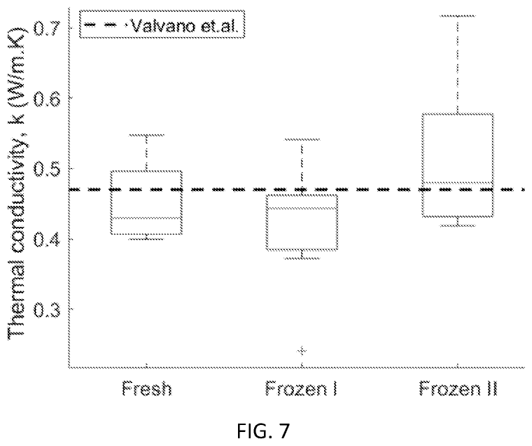

The reflectance standard used in this experiment includes a highly reflective surface which was considered as 100% reflection. All measurements from tissue and glass surfaces were corrected according to the reflectance standard reading. FIG. 7 shows different measurement techniques used in this test. Finally, the control experimental results were taken into account for initial measurement of R_{sample+glass} to calculate the actual reflectance from the tissue surface, as shown in Equation 7. A total of 14 samples were used for this test, which included both fresh (n=7) and frozen (n=7) tissue samples.

$${M}_{R} ( \% ) = ( \frac{{R}_{samp1e + glass} - {R}_{glass} - {R}_{0}}{{R}_{standard} - {R}_{0}} ) * 100 \qquad ( 7 )$$

After evaluating the experimental transmittance and reflectance data, the absorbance and reduced scattering coefficients (μₐ, μₛ′) were estimated through IAD. This computational approach utilizes a Monte Carlo simulation technique focusing on the integrating sphere setup [34]. The current study utilized the most recent version of the program developed by Prahl and Jacques. The open-course source code is written in C and has been adopted in other tissue optical studies [36, 37]. As an input, the program requires tissue specific properties such as index of refraction, sample thickness, experimental light transmittance, and experimental reflectance values, among others. It also requires the dimensions of the integrating sphere used in the experiment (number of spheres used, size, port diameters etc.), the laser beam area, and wavelength. All these input parameters were measured during the above-mentioned experiments with the exception of the refractive index, which was obtained from literature (1.34˜1.37 for porcine pancreas tissue) [21, 38]. As two measurements were taken during this optical study (M_{T} and M_{R}), the anisotropy, g=0.85 was assumed for the IAD run as it is typical for the biological tissue in NIR range [39,40]. Once the input parameters were set, the IAD code would generate a list of output results including the μₐ, μₛ′, anisotropy (g), and penetration depth which were substituted into equation 2 to evaluate the attenuation coefficient (μ_{eff}) for both fresh and frozen samples.

### B. Result and Discussion

Thermal Conductivity Test Results. To validate the experimental setup, two different controls were measured: a polymer of known thermal conductivity and chicken breast samples. The heat transfer coefficient for free flow of air can be anywhere between 0.5 and 1000 Wm⁻² K⁻¹ [39]. Therefore, using the known thermal conductivity of PDMS, the experimental heat transfer coefficient of the system was determined to be approximately h=20 Wm⁻²K⁻¹ Furthermore, an experimental range of temperatures between 35° C. and 45° C. was determined using samples from the chicken specimen. Additionally, these preliminary experiments helped determine an ideal sample thickness of ˜1 cm.

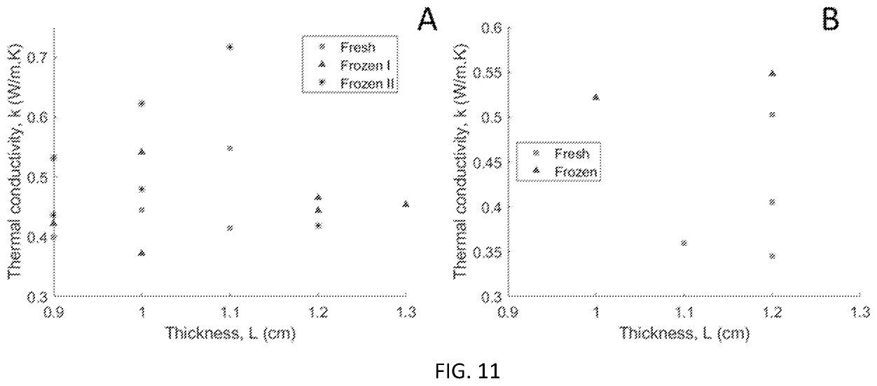

Assessment of the impact of freezing/thawing on thermal conductivity for porcine pancreas tissue was completed through comparison with freshly obtained samples. These experiments were conducted for two different temperatures: one within the previously established experimental range (40° C.) and the other at a hyperthermic level (50° C.) (see FIG. 11). For the experiments at 40° C., 17 samples were utilized: 5 were used same day as fresh samples and the other 12 were frozen for two different durations. The first group of frozen specimens (Frozen I) contained 6 samples which were kept frozen for 7 days after excision. The second group of frozen specimens (Frozen II) had 6 samples which were kept frozen for 14 days after excision. A similar study was conducted at 50° C. where two types of tissue samples were utilized: fresh (n=5), and frozen (n=6, frozen for 14 days). Note that the frozen samples were only thawed once, as the degradation of the tissue after a second freezing cycle rendered an almost-liquid sample.

The measurements at 40° C. yielded similar yet dispersed values for the frozen and fresh groups (FIG. 7). After testing for normality and with a 14% coefficient of variation, the data set was considered to follow a normal distribution and Chauvenet's criterion was implemented to identify outliers. Careful examination of these outliers supported the previous observation, as the outlying samples were observed to exhibit structural deterioration or excess fatty tissue, influencing the overall behavior of the thermal properties. After removing the outliers, the resulting data sets show no significant difference (T test, P=0.893) between the thermal conductivities for fresh specimens (k_{fresh}=0.451±0.067 Wm⁻¹ K⁻¹), and frozen specimens (k_{frozen}_{_{I}}=0.449±0.056 Wm⁻¹ K⁻¹ k; K_{frozen}_{_{II}}=0.466±0.05 Wm⁻¹ K⁻¹). These results are in agreement with the value of 0.47 Wm⁻¹ K⁻¹ reported by Valvano et al for porcine pancreas at 40° C. [18]. The average thermal conductivity at 50° C. for fresh and frozen samples were measured at 0.503±0.071 and 0.535±0.019 Wm⁻¹ K⁻¹, respectively, which were higher than the results obtained at 40° C.

It is important to note that the measured thermal conductivity of fresh porcine pancreas tissue (0.451 Wm⁻¹ K⁻¹) was lower than that of porcine liver tissue (0.53 Wm⁻¹ K⁻¹), as the latter has been assumed for pancreas tissue in other studies [18]. Another critical finding was that the measured thermal conductivity of fresh and frozen samples (7 days) were not significantly different. Though the variation of extended frozen samples (14 days) were higher than the fresh samples, the mean values were close for both tissue types. It is recommended not to use long frozen samples at higher temperatures (≥40° C.) for applications which are dependent on thermal conductivity of the tissue because this thermal property tends to increase with temperature. Pathological analysis is required to identify the changes in microstructure due to freezing and defrosting. Apart from that, tissue sample preparation, convective heat transfer rate, thermal probe sensitivity, as well as the differences between the current experimental setup and the experimental setup utilized by Valvano et al., might have introduced measurement artifacts and variation within results.

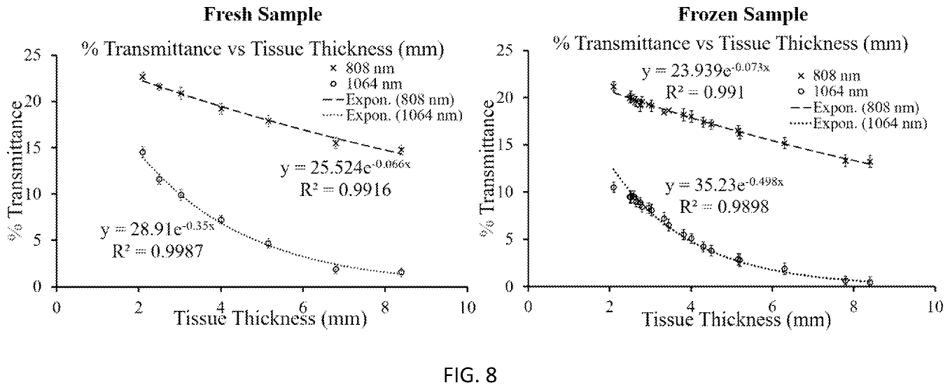

Light Transmittance and Reflectance Test Results. In this test, the light transmittance and reflectance of fresh and frozen tissue samples were measured for both 808 and 1064 nm light. Laser intensity was fixed at 5 mW·mm⁻² for all test conditions. These results were further utilized to evaluate different optical properties including μₐ and μₛ′ (unit: mm⁻¹) through IAD simulation. FIG. 8 shows the transmittance vs tissue thickness graph for fresh (n=7) and frozen samples (n=17). Tissue thickness measured by ImageJ were in the range of 2-9 mm. Each data point in the graph represents an average of five readings taken from the same tissue thickness. The error bar represents ±1σ (standard deviation). Statistical analysis (T-test) among data for fresh vs frozen tissue samples showed P<0.0001. Light transmittance at 808 nm wavelength was found to be higher than that at 1064 nm for both fresh and frozen samples as predicted from literature [7]. One of the key findings of this experiment was the distinction between two different tissue samples: fresh samples showed slightly higher light transmittance than frozen samples at both wavelengths. For 2.5 mm thick tissue layers, the average light transmittance values through fresh samples at 808 and 1064 nm wavelengths were 10.6±2.4% and 21.4±2.9% higher than those for frozen samples, respectively. This variation can be attributed to the physiological change in tissue properties with respect to time, which ultimately affected the light transmittance, as water absorption peak gradually increases in the mid IR range. MATLAB's curve fitting tool was used to fit the dataset, which demonstrated an exponential decay of light transmittance with respect to the increase of tissue thickness for both fresh and frozen samples (R²>0.98).

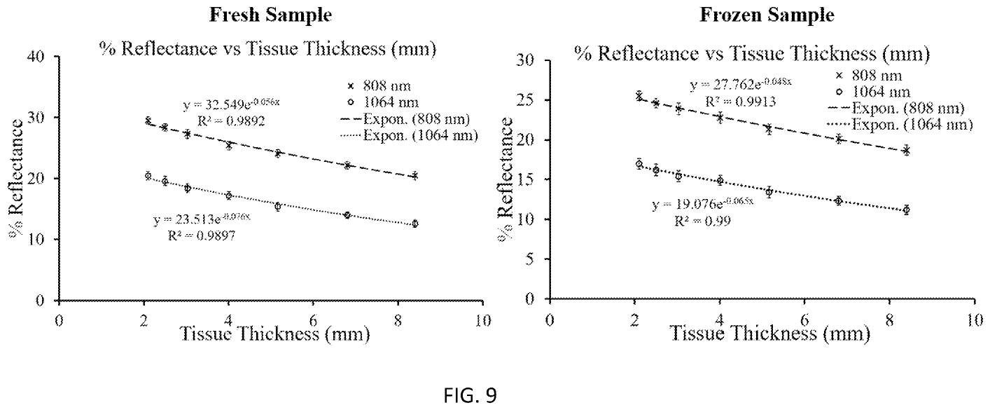

Next, the light reflectance was measured for both fresh (n=7) and frozen (n=7) tissue samples (tissue thickness 2-9 mm) and laser wavelengths. FIG. 9 shows the result of this test where each data point represents the mean reflectance value of five repeated tests from the same tissue sample. The error bar represents ±1σ (standard deviation). Like the previous test, statistical analysis (T-test) depicted the significance of the results (P<0.0001) when fresh and frozen samples were compared. The results of this test showed a gradual decrease of reflectance value with the increase of tissue thickness for all test conditions. The 808 nm had higher reflectance than 1064 nm for both types of samples. Fresh tissue samples displayed higher reflectance than frozen samples (approximately 15 to 21% higher for both laser wavelengths). For a 2.5 mm thick tissue samples, the light reflectance at 808 and 1064 nm were 28.4±2.7% and 19.6±2.5% for fresh samples and 24.6±1.8% and 16.2±1.3% for frozen samples, respectively.

The absorption and reduced scattering coefficients of porcine pancreas tissue were computed through IAD. This method requires the experimental results of light transmittance and reflectance test as inputs. The remaining input parameters, including tissue thickness and physical dimensions of integrating sphere, were measured during the previous optical tests. IAD was run for different tissue thicknesses including both fresh and frozen samples. The results of this test are listed in following Table 1 along with the porcine liver optical properties obtained from Ritz et al. (2001) [40].

TABLE 1
Porcine pancreas tissue optical properties obtained from
IAD and a comparison with porcine liver properties.

| | | | Reduced |
|---|---|---|---|
| | Wave- | Absorption | Scattering |
| | length | coefficient, μₐ | coefficient, μₛ′ |
| Tissue Type | (nm) | (mm⁻¹) | (mm⁻¹) |
| Fresh porcine pancreas | 808 | 0.016 ± 0.009 | 0.120 ± 0.011 |
| | 1064 | 0.059 ± 0.010 | 0.792 ± 0.008 |
| Frozen porcine pancreas | 808 | 0.020 ± 0.007 | 0.134 ± 0.010 |
| | 1064 | 0.095 ± 0.005 | 0.951 ± 0.009 |
| Porcine liver³⁹ | 830 | 0.073 ± 0.004 | 0.385 ± 0.350 |
| | 1070 | 0.018 ± 0.001 | 0.304 ± 0.170 |
| Frozen human | 1064 | 0.090 ± 0.010 | 2.340 ± 0.010 |
| neuroendocrine pancreas | | | |
| (tumor)¹⁵ | | | |

From Table 1, it can be noted that both absorption and reduced scattering coefficients of porcine pancreas tissue showed an increasing trend from fresh to frozen at both laser wavelengths. At 808 nm, μₐ and μₛ′ increased by 16.7±6.1% and 10.2±3.7%, respectively, for the frozen samples. For similar tissue conditions at 1064 nm, μₐ and μₛ′ increased by 60.7±5.4. % and 20.1±4.8%, respectively. Comparison of these optical properties between the fresh porcine pancreas tissue measured in this study versus liver tissue properties in Ritz et al. showed significant variation (T-test, P<0.0001) (39). Pancreas tissue displayed higher absorption and scattering coefficients at 1064 nm wavelength when compared to liver tissue at 1070 nm. At lower wavelengths (808/830 nm), an inverse trend was observed. This deviation could be attributed to the difference in tissue morphology, which clearly distinguishes liver tissue optical properties from pancreas tissue. This is a critical finding of this study, as many groups have assumed these liver tissue optical properties for pancreas tissue. In addition, optical properties of human neuroendocrine tumor of pancreas were compared against porcine pancreas at 1064 nm. These two different tissue types showed a significant variation in μₛ′ (2.34 vs 0.788 mm⁻¹) when compared to μₐ (0.09 vs 0.06 mm⁻¹) which can be attributed to the significantly different tissue morphology of both tumor type.

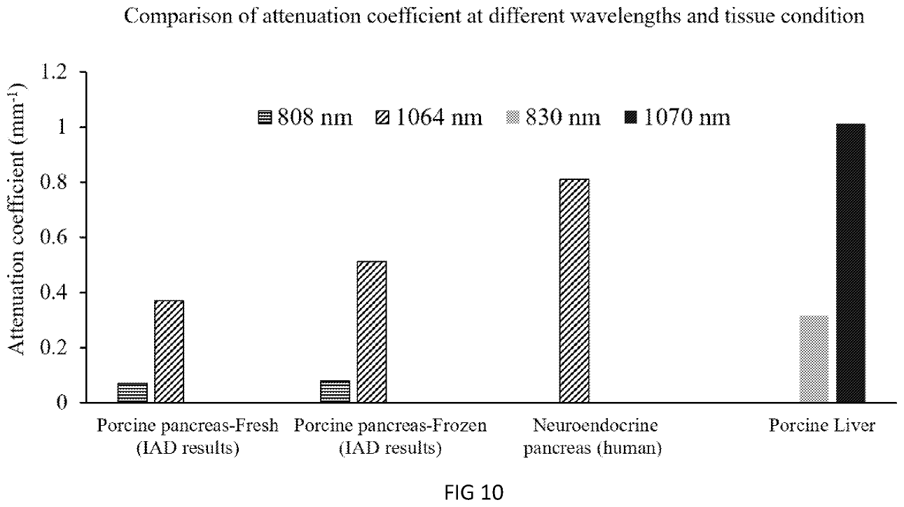

The absorption and reduced scattering coefficients obtained from IAD were utilized to evaluate the attenuation coefficient (μ_{eff}, unit: mm⁻¹) by using Equation 2 which is reported in Table 2. Also, the same optical property was compared against porcine liver tissue and human pancreas neuroendocrine tumor as reported in literature. These results are shown in FIG. 10.

TABLE 2
Light attenuation coefficient at 808 and 1064 nm wavelengths
for fresh and frozen porcine pancreas tissue samples

Attenuation coefficient (μ_{eff}) (mm⁻¹)

| | Fresh Sample | Frozen Sample |
|---|---|---|
| 808 nm wavelength | 0.081 ± 0.005 | 0.096 ± 0.007 |
| 1064 nm wavelength | 0.389 ± 0.010 | 0.545 ± 0.004 |

It is evident from FIG. 10 that attenuation coefficient differs significantly (T-test, P<0.001) from fresh to frozen samples: 13.04% and 37.8% at 808 and 1064 nm wavelengths, respectively. This variation can be attributed to the rapid onset of autolysis or autodigestion [41]. Pancreas tissue starts to deteriorate quickly after dissection due to the loss of enzymes and other biofluids [42]. The lobular structure of the tissue is clearly evident in fresh samples but was rarely visible in the frozen samples.

The results provided by the IAD method might be affected by the error involved in the light transmittance and reflectance experiments. The variation in experimental results could be attributed to some of the measurement methods used in this study. The ImageJ technique is reliable but depends on the image quality and the view angle. A slight change in focus angle might cause a variation in the measurement of tissue thickness. The glass holder design for securing the tissue sample had four adjustable spring-loaded screws to avoid excess pressure on the tissue. A slight change in the turning of the screws could misalign the top glass slide and tilt the surface of the tissue. This variation in alignment was identified and corrected using ImageJ software by analyzing the tissue side view image. The integrating sphere and the photodetector used in this study both have a tolerance limit, and it should be noted that both were calibrated and certified by the manufacturer before using in the experiment.

Despite having some limitations, the experimental technique utilized in this study more rigorous than many studies reported in literature. For example, a double integrating sphere technique was described by Saccomandi et al. (2016) wherein a fiberoptic probe was utilized for illuminating the human neuroendocrine pancreas tumor and measuring the light transmission and reflection. This group utilized an inverse Monte Carlo simulation technique to evaluate the tissue optical properties. This technique allowed the transmitted photons through the tissue sample to reflect due to the internal reflection from the inner surface of the integrating sphere. Light transmission and reflection through the tissue sample continued until it reached a steady state. Although the double integrating sphere technique is less time consuming as both light transmission and reflection data can be collected simultaneously, the accuracy is dependent on proper experimental technique as described by Prahl et al. [32]. Prahl described that collimated light is necessary for optical property measurements in tissue. Light from a fiberoptic tip emits in a cone dictated by the numerical aperture of the fiber, which can increase measurement error in an integrating sphere. This factor should be considered while collecting the data, and the IAD approach needs to be corrected accordingly to minimize the error involved in the numerical result. The current study employed a collimated beam and a single integrating sphere technique to avoid such error that could impact the accuracy of experimental and numerical results of the tissue optical properties.

FIG. 10 also compared the optical properties of porcine pancreas with liver tissue [40]. It shows a significant difference in attenuation coefficient for both laser wavelengths (79.1±0.3% and 65.4±0.5% higher at 808 and 1064 nm wavelengths, respectively, when compared to the experimental values obtained from fresh porcine pancreas tissue), which proves the necessity of using correct optical properties while performing computational modeling or experimentation with porcine pancreas tissue. Assuming liver tissue optical properties for pancreas tissue would cause a high degree of error that could undermine a model or therapeutic procedure. Porcine pancreas tissue optical properties were rarely evident in the literature, which is a significant gap due to its being the most frequently used animal model for human pancreas. The current study has helped to fill this research gap in addition to motivating further development of PPTT for pancreatic cancer treatment.

An innovative fiberoptic microneedle device (FMD) developed by this group is capable of co-delivering photoabsorbers in solution (GNRs) and high intensity light (NIR) to a targeted tissue volume [43-45]. In addition, the sharp, needle geometry enables penetration through soft tissue to emit proximal to a malignant target, reducing damage to healthy tissue in the optical path. Local photothermal heating demonstrated a higher degree of penetration and controlled volumetric dispersal of macromolecules in rat cerebral and porcine bladder tissue [10, 46]. These phenomena inspired the application of PPTT using FMD for the treatment of pancreatic cancer.

Testing and validation of thermal conductivity setup. To validate the setup, we measured the thermal properties of a known material, PDMS. The polymer sample was tested at a temperature of 55° C. (n=1), greater than the maximum testing temperature, to characterize the air flow in the system and account for any changes that may arise from utilizing different temperatures. Samples from the chicken specimen were utilized to validate the experimental setup at two different temperatures: 35° C. (n=5) and 45° C. (n=9) to ensure a working range of temperatures for the setup. The pancreas tissue samples were tested at 40° C. (n=16) and 50° C. (n=7) to identify differences between measurements inside and outside the working temperature range.

The thermal conductivity of Sylgard™ 184 is reported as 0.27 Wm⁻¹ K⁻¹ [28]. Solving for k in Equation 1, the measured thermal conductivity of PDMS at 50° C. was ˜0.25 Wm⁻¹ K⁻¹ when h=20 Wm⁻²K⁻¹. Thus, the experimental heat transfer coefficient was determined to be 20 Wm⁻²K⁻¹. To further validate the setup, measurements using chicken breast samples were conducted and compared to available published values. The reported thermal conductivity of chicken breast, at temperatures below 40° C., ranges between 0.45 and 0.5 Wm⁻¹ K⁻¹ [29, 47]. The two different temperatures were selected to both compare with published data (35° C.) and to examine the effects outside of the published data range (45° C.). The thickness of the samples utilized in these tests ranged between 1-1.5 cm. The outliers found in the data were associated to limitations in the sensitivity of the experimental setup, as thicker samples yielded less accurate results, and Chauvenet's criterion was employed to remove these outliers. It was found that the measurements at 35° C. yielded a thermal conductivity in agreement with the published data (0.440±0.029 Wm⁻¹ K⁻¹). Interestingly, the measurements at 45° C. (0.461±0.041 Wm⁻¹ K⁻¹) also fall within the range established in published literature. From these experiments, a clear relationship between thickness and thermal conductivity was not determined. Additionally, to avoid systemic errors in the experiments with pancreas tissue samples, the ideal sample thickness was determined to be ˜1 cm.

The data obtained from measuring the thermal conductivity of pancreas samples is shown in FIG. 11.

Knowledge of the thermal and optical properties of tissue is a foundational necessity for developing innovative therapies such as PPTT. However, properties of pancreas tissue have rarely been explored, especially in the NIR region, and are often assumed to be those of liver tissue. This work characterized the thermal conductivity, optical reflectance, and optical transmittance of healthy porcine pancreas tissue using both fresh and frozen samples. The thermal conductivity was measured at 40° C. and 50° C., and the reflectance and transmittance were measured under 808 and 1064 nm wavelengths. Interestingly, pancreas tissue thermal properties when demonstrated to be conserved after freezing for up to three weeks. Conversely, pancreas tissue optical properties underwent significant change after freezing, suggesting that fresh samples should be used for optical property dependent experimentation. These findings can be used to inform photothermal damage models and in assessment of thermal therapy effects in pancreas tissue. Furthermore, characterization of healthy tissue properties will provide more insight and aid in successfully understanding the effects observed during treatment in both healthy and cancerous tissue.

### Example 2

### Image-Guided Theranostic Approach for Pancreatic Cancer Based Upon Plasmonic Photothermal Heating

Experimental Measurements of Tissue Properties. In order to predict the extent of photothermal heating in pancreatic tissue, properties including the thermal conductivity of the tissue and its optical transmissivity must be well-characterized; these properties were previously lacking in the literature. The inventors have measured the thermal conductivity of ex vivo porcine pancreas samples and determined it to be approximately 0.45 W/m·K. The inventors also measured the optical attenuation of ex vivo porcine pancreas at 1064 nm to be approximately 0.075 cm⁻¹ (43).

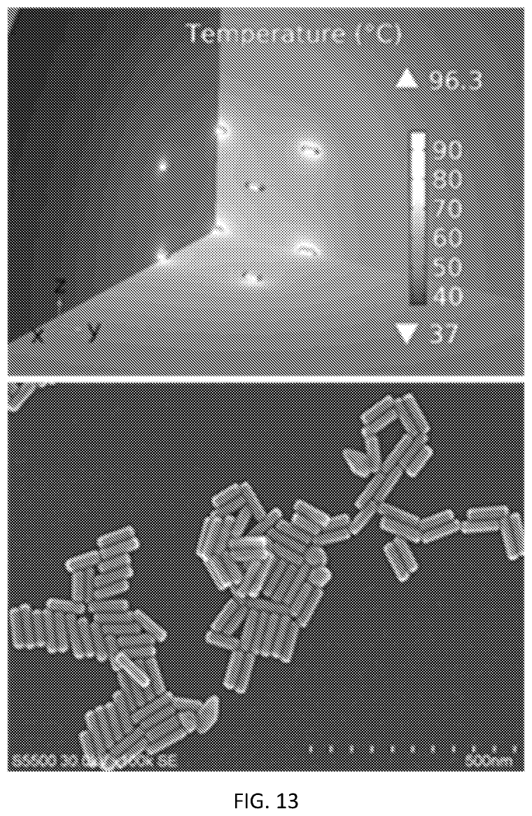

PPTT Multiphysics Modeling. The inventors have developed a multiphysics computational model to study the optical properties of GNPs and the subsequent plasmonic heating due to light-particle interactions. FIG. 13(a) shows an example of the results of the multiphysics model. In this example, a cluster of 27 gold nanorods (GNRs) arranged in a three-dimensional cubic array was modeled. The optical absorbance of the cluster was calculated using COMSOL®'s RF module, a finite element method (FEM) solver of Maxwell's equations of electrodynamics. The absorbed energy was then used as the heat source in an FEM model of the heat transfer equation, resulting in the temperature map shown in FIG. 13(a) (13). FIG. 13(b) shows an electron micrograph of the as-synthesized GNRs.

Fiber Optic Microneedle Device. The fiber optic microneedle device (FMD) is a microneedle catheter capable of penetrating soft tissues and co-delivering laser light and fluid agents (FIG. 14). The FMD was adapted to enhance the volumetric dispersal of macromolecules delivered to the brain through convection-enhanced delivery (CED) by concurrent delivery of sub-lethal photothermal hyperthermia (45, 57). For this, FMDs were inserted into both cerebral hemispheres of anesthetized rats to a depth of 1.5 mm. Laser irradiation (1064 nm) at 0 (control), 100, and 200 mW was administered concurrently with CED infusions of liposomal rhodamine (LR) or gadolinium-Evans blue-serum albumin conjugated complex (Gd-EBA). Brains treated with 200 mW co-delivery exhibited thermal lesions, while 100 mW co-deliveries were associated with preservation of brain cytoarchitecture (44, 45). Subsequently, it was demonstrated that the volumetric dispersal of infused contrast agents into healthy rodent cerebral tissue was enhanced by the FMD and scaled with increasing laser power up to 200 mW (44, 45).

Plasmonic GNPs. Synthesis and characterization of plasmonic GNPs, as well as development of bio-applications of these particles, is well established in the Mayer group. The inventors recently described the optical properties of GNRs for photothermal heating applications (58). The inventors also recently performed an assessment of two types of biocompatible GNPs with different surface coatings (citrate and polyethylene glycol) for another medical application (radiation therapy) (59, 60).

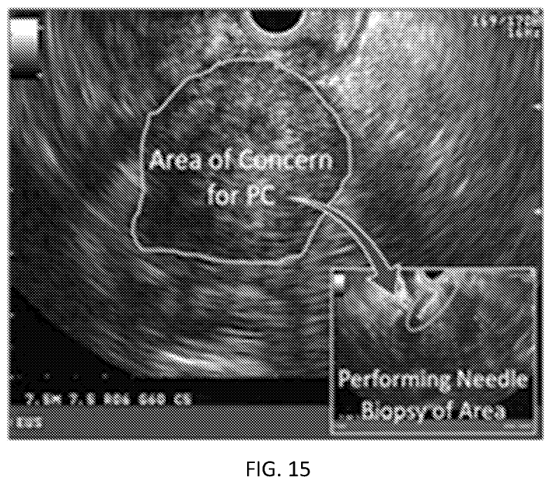

Real-time Imaging of Pancreas via Endoscopic Ultrasound. EUS has been performed to diagnose pancreatic diseases and cancer. The inventors developed expertise in using stress/strain elastography to improve the diagnosis of PC via ultrasound (61). FIG. 15 shows an example of EUS images of taking a biopsy of cancer in the head of the pancreas obtained during a procedure in a clinic.

Preparation and functionalization of gold nanoparticles. Gold nanorods (GNRs) are chosen due to their ease of synthesis and functionalization and their high absorbance cross-section in the nIR, which leads to efficient photothermal heating. GNRs are predicted by computational model to be capable of localized hyperthermic heating (13); this has also been demonstrated experimentally in vitro and in vivo by El-Sayed et al. (12, 62). GNRs will be synthesized by a seed-mediated growth protocol described in the literature (63-65). Optical absorbance of the nanorods will be confirmed via UV-Vis spectroscopy and their geometry (length, width, aspect ratio) will be measured via SEM and analyzed in ImageJ.

Both targeted and non-targeted GNRs will be tested. Nontargeted GNRs will be functionalized with polyethylene glycol (PEG), a biocompatible polymer (66). Briefly, carboxy-PEG-thiol will be added to a solution of as-synthesized GNRs. Over 24 hours, the PEG displaces the native surfactant layer on the GNRs, after which the PEGylated GNRs are washed and resuspended in aqueous solution. Targeted GNRs will be conjugated with antibodies with binding specificity for biomarkers which are overexpressed in cancer types including PC, e.g. EGFR (epidermal growth factor receptor) (12, 67). To produce the antibody-conjugated GNRs, as-synthesized GNRs will first be functionalized with a self-assembled monolayer (SAM) of undecanoic acid, creating a carboxy-terminated surface. The particles will be washed and suspended in buffer at pH 6.8. Targeting antibodies will be added along with EDC (1-ethyl-3-(-3-dimethylaminopropyl) carbodiimide hydrochloride) to initiate carbodiimide cross-linking (68). The nanoparticles will then be washed and resuspended in buffer. The targeting antibody will be monoclonal mouse anti-human EGFR antibodies obtained from Thermo Fisher/Invitrogen. PEGylated and antibody-conjugated GNRs, as well as the as-synthesized GNRs, will be characterized via dynamic light scattering and zeta potential measurements in order to determine their size distribution and surface charge. The function of surface-bound antibodies will be assessed via the plasmon resonance shift upon target binding using UV-Vis spectroscopy.

Characterizing gold nanoparticle infusion in tissue. FMD will be utilized to infuse PEGylated GNRs in a pancreas tissue phantom, and freshly dissected porcine pancreas (ex vivo study). For biorepository samples (ex vivo human pancreas tumor samples), both targeted (antibody-conjugated) and nontargeted (PEGylated) GNRs will be tested and the localization of GNRs within the tumor will be compared for both preparations. It is contemplated that GNR delivery accompanied with heating to sub-hyperthermic temperatures (˜40° C.) via IR laser illumination at 1064 nm over a clinically realistic (˜10 min) duration will statistically significantly improve the volume of dispersal as compared to delivery without laser heating. We further hypothesize that antibody targeting will significantly improve the percentage of GNRs localized inside the tumor as compared with polymer-coated GNRs.

Ex vivo Tissue Phantom Experiment. The tissue phantom will be prepared from agarose-based hydrogel (69, 70) to mimic the acoustic and diffusive properties of pancreas tissue (71, 72) and the nanoparticle diffusion process (73). Tissue phantoms will be formed into rectangular prisms of 2×2×1 cm³ and maintained at a range of experimental temperatures 10-40° C. Microneedles will be inserted into the phantoms to a depth of 5 mm and PEGylated GNRs will be infused at 10-100 μL/min for 10 min. The concentration of the GNRs will range from 0.5-10 nM, a sufficiently high value so that it can be detected visually and via ultrasound (US). The latter will utilize a high resolution, fast response system (Mindray M6 laptop-style with DICOM export capability and an L14-6 s (linear 14-6 MHz) probe). Validation in tissue phantoms will enable the use of this US approach for measuring volumetric dispersal in pancreas tissue.

Ex vivo porcine pancreas tissue experiments. Ex vivo porcine pancreas will be harvested from a USDA approved abattoir. Experiments will investigate the effects of photothermal heating on volumetric dispersal and distribution of GNRs. The approach will follow methods described in Hood et al. (45). Briefly, pancreas tissue will be heated with a 1064 nm continuous wave (CW) laser prior to and during infusion. Laser parameters and fluid flow rates will be experimentally varied within the ranges of 0-20 mW/mm² and 0-1000 μL/min, respectively, as shown in Table 3. The zero values represent experimental controls. Timeframes will not exceed 30 minutes, as this is the upper range allowable to maintain clinical utility. For these experiments, tissue samples roughly analogous in size to the previously mentioned tissue phantoms will be employed. The FMD will be used to irradiate the tissue and infuse the GNR solution. Preliminary studies with porcine pancreas tissue demonstrated that an irradiance of 2.8 mW/mm² with a 1064 nm CW laser increased tissue temperature by 2.2° C. at 3 mm depth from the top surface of the tissue over 5 minutes. It is expected that tissue temperature will be increased up to 10° C. to assess impact on the volumetric dispersal of infused GNRs. Volumetric distribution of GNRs will be measured at 1-minute intervals with US, and DICOM images will be captured and transferred via USB for comparison with pathology analysis. After experimental infusion and irradiation is complete, tissue samples will be formalin-fixed, stained with hematoxylin and eosin, and gross sectioned for whole mount examination. Samples will then be sectioned via microtome for more detailed evaluation. All sequential tissue sections will be imaged under brightfield microscopy and the volume of infusion measured through image thresholding of 2D areas and interpolation of the 3D volume in MATLAB.

TABLE 3
Experimental Parameters for GNR Infusion in Tissue

| Parameters to be tested | | Primary measurement from the test |
|---|---|---|
| 1064 nm | 0-20 mW/mm² | Tissue temperature increase (ΔT, ° C.), |
| CW Laser | Irradiance will be | Volumetric dispersal of infusate (mm³), |
| Power | increased gradually | Thermal injury of tissue volume (mm³) |
| | by 1 mW/mm² | |
| Laser | Up to 10 min. | Tissue temperature increase (ΔT, ° C.), |
| duration | Cut off if ΔT = 10° | Volumetric dispersal of infusate (mm³), |
| | C.; gap time 30 s | Thermal injury of tissue volume (mm³) |
| Infusion | 10-1000 μL/min, | Perpendicular infusate penetration (mm) |
| rate | Infusion rate will be | |
| | increased gradually | |
| | by 10 μL/min | |

Ex-Vivo Human Tissue Experiments. Human PC samples will be obtained from the UT Health San Antonio Biorepository. Samples are regularly harvested by hepato-pancreatico-biliary surgeons during resection of the cancerous gland in procedures such as Whipple's and tail resections. Whole specimens will be acquired and sectioned for testing of injection and ablation experiments as described previously. Laser and infusion parameters will be selected based on previous results to demonstrate maximum effect on volumetric dispersal without coagulating tissue. Dispersal volume of both PEGylated and anti-EGFR conjugated GNRs will be monitored by US. After experimentation, samples will be fixed in formalin and sectioned by a trained pathologist as described above. Samples will then be examined using optical microscopy to ascertain the final dispersal volume of the infusate, as well as investigate localization of GNRs.

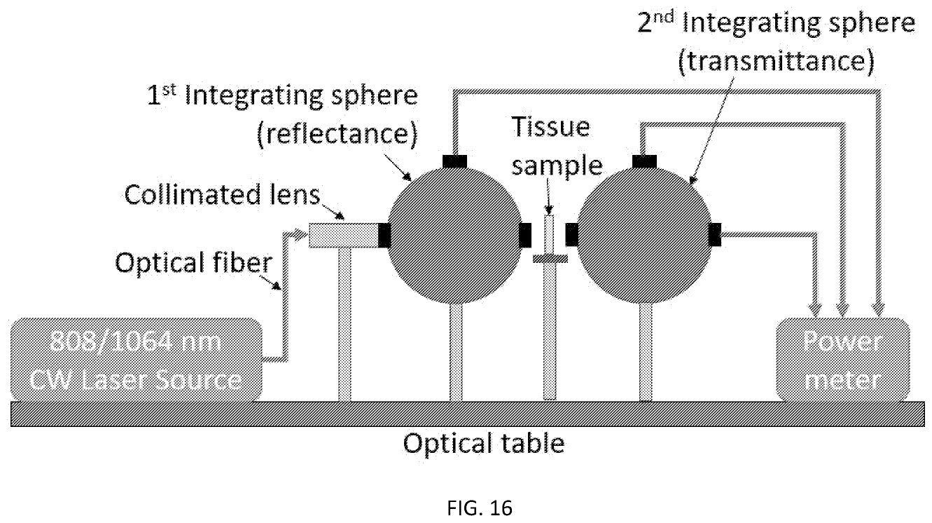

Pancreas tissue properties characterization. The current characterization of optical properties for pancreas tissue, both healthy and cancerous, is highly incomplete. While some measurements have been made at 1064 nm by this group and others (43, 15), none have been made for 808 nm, which is the intended excitation wavelength for the GNRs. The proposed study will follow methods described in Saccomandi et al., which employed a dual integrating sphere system to characterize optical properties in pancreas tissue (15). In this approach, two photodetectors are placed on either side of a 1 mm thick pancreas tissue sample as shown in FIG. 16. A laser is transmitted through the first integrating sphere to illuminate the tissue sample. The first photodetector measures reflected light from the surface of the sample, and the second photodetector measures transmitted light. Based on these measurements, the inventors can obtain scattering and absorbance coefficients and validate the approach against previously measured values for 1064 nm before moving on to 808 nm. Experiments will be conducted at 808 and 1064 nm to characterize optical properties in various pancreas tissue types, including ex vivo porcine as well as healthy and cancerous human tissue from the biorepository.

Computational modeling. Implementation of theranostic approaches such as PPTT requires the ability to reliably predict the effect of the plasmonic heating on the surrounding tissue. To that end, a computational model that accurately represents the laser-nanoparticle-tissue interactions is needed. This group has previously developed computational models to study optical absorption of GNRs at the nanoscale (13). These models showcase the effects of individual and arrays of GNRs under laser illumination, described as an infinite plane wave, and homogeneous surrounding medium properties (e.g., water). At the tissue level, the light-nanoparticle-tissue interactions behave differently than at the nanoscale. The proposed model includes three components: Maxwell's equations solver that computes the heat sources generated by light-nanoparticle interaction, bioheat transfer solver that computes temperature field in the tissue, and ablation volume solver that is based on both CEM 43 thermal dosage and Arrhenius thermal damage model (74-76). The proposed computational model will also incorporate the tissue optical properties characterized as described above. Parameters for a realistic light source, similar to the one used in the experiments, will be included in the model. The spatial distribution of GNRs will be modeled based on the data collected. Clusters of GNRs will be represented as point sources with a given power. The specified power will be associated to the gold density assigned to each point source. The results of the proposed computational model can be compared and validated with the measurements obtained. This model can serve as a predictive tool, allowing us to examine the extent of tissue damage caused by the therapy.

Measurement of thermal ablation zone. While non-plasmonic laser ablation (i.e. LITT) of PC has been demonstrated previously, issues with broad, non-specific damage and subsequent effects such as pancreatitis limit the technique's utility. The proposed approach using PPTT is designed to overcome these drawbacks through leveraging functionalized GNRs excited at 808 nm, which has very low optical interaction in human tissue. Due to the localization of GNR-mediated photothermal ablation, PPTT has strong potential for improved healthy tissue sparing as compared with other techniques. In addition, due to the strong ultrasound contrast of GNRs, the volume infused with GNRs can be imaged via US. It is contemplated that this capability paves the way for an image-guided theranostic modality in which the GNR spatial distribution is used to precisely predict the resulting zone of ablation. For this ex vivo study of the thermal ablation resulting from PPTT, it is contemplated that the volume of the ablation zone will match the volume infused with GNRs as described by ultrasound imaging, to within the resolution limitations of the US.

Tissue phantom thermal ablation. Tissue ablation experiments will follow methods previously described by this and other groups (77-79). To characterize the performance of PPTT, laser irradiation experiments will be conducted at 808 nm with the tissue phantom as described. Laser powers in the range of 10-150 mW/mm² will be assessed for irradiation times of 0-15 min. Each set of parameters will be used to irradiate small tissue samples (larger than the beam width) soaked with GNR solutions of 0.5-10 nM concentration overnight. Photothermal heating will be assessed with infrared thermometry and thermocouples within the tissue (outside the beam path). Ablation volumes will be assessed using pathology methods.

Infused tissue thermal ablation. A series of experiments in ex vivo porcine pancreas tissue will be conducted to determine the PPTT-induced zones of ablation and necrosis due to PEGylated GNRs. Ex vivo porcine tissue samples will be prepared as stated previously. An FMD will be advanced 5 mm into the tissue sample. PEGylated GNRs in solutions at concentrations ranging from 0.5-10 nM will be infused at 10 μL/min for 15 minutes while being tracked under US imaging. Laser irradiation at 808 nm will be provided through the FMD. Laser powers in the range of 10-150 mW/mm² will be assessed for irradiation times of 0-15 min. A head-to-head comparison between PPTT and LITT will also be carried out. For the LITT comparison, illumination at 1064 nm in the absence of GNRs will be applied via the FMD. In all cases, photothermal heating will be assessed with infrared thermometry and thermocouples within the tissue (outside the beam path). Ablation volumes will be assessed through tissue fixation, sectioning, and inspection by a trained pathologist. GNR infusion volume will be assessed from the US images and verified through pathology imaging. An additional series of experiments will be conducted using patient tissue samples from the biorepository. Due to the scarcity of tissue, experimental parameters identified during experiments with the porcine tissue will be utilized. Experiments will examine and compare the localization and defined ablation boundaries of infused PEGylated GNRs, anti-EGFR conjugated GNRs, and saline solution (negative control) within both healthy and cancerous tissues. Again, a comparison with LITT (no GNRs; illumination at 1064 nm) will be carried out as described above.

### Example 3

### Assessment and Modeling of Plasmonic Photothermal Therapy Delivered Via a Fiberoptic Microneedle Device Ex Vivo

Plasmonic photothermal therapy (PPTT) has potential as a superior treatment method for pancreatic cancer, a disease with high mortality partially attributable to the currently non-selective treatment options. PPTT utilizes gold nanoparticles infused into a targeted tissue volume and exposed to a specific light wavelength to induce selective hyperthermia. The current study focuses on developing this approach within an ex vivo porcine pancreas model via an innovative fiberoptic microneedle device (FMD) for co-delivering light and gold nanoparticles. The effects of laser wavelengths (808 vs. 1064 nm), irradiances (20-50 mW·mm⁻²), and gold nanorod (GNR) concentrations (0.1-3 nM) on tissue temperature profiles were evaluated to assess and control hyperthermic generation. The GNRs had a peak absorbance at ˜800 nm. Results showed that, at 808 nm, photon absorption and subsequent heat generation within tissue without GNRs was 65% less than 1064 nm. The combination of GNRs and 808 nm resulted in a 200% higher temperature rise than the 1064 nm under similar conditions. A computational model was developed to predict the temperature shift and was validated against experimental results with a deviation of <5%. These results show promise for both a predictive model and spatially selective, tunable treatment modality for pancreatic cancer.

### A. Results

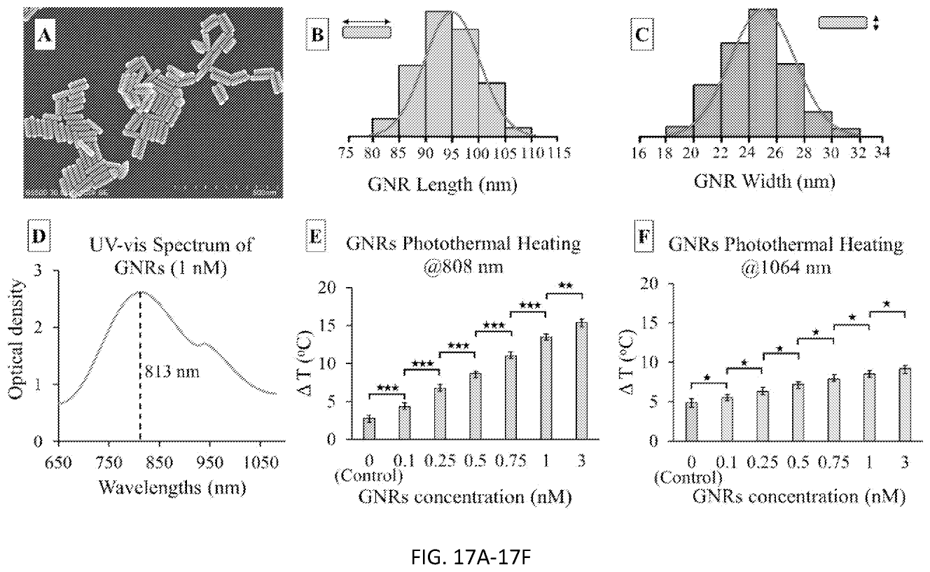

GNRs Synthesis and Photothermal Heating. SEM images of the GNRs were utilized to obtain the average dimension of nanorods (FIG. 17). The measured length and width of the GNRs were 95.2±4.7 nm, and 24.8±1.5 nm respectively. Spectrometric analysis showed the resonance wavelength (peak absorbance) of the GNRs to be 813 nm. At 1064 nm, the GNRs optical absorbance reduced to less than half of the peak absorbance. Theoretically, these GNRs should exhibit high photon absorption and heat generation at 808 nm which was assessed in this experiment. The photothermal heating experiment of different GNR concentrations (0.1-3 nM) at 808 and 1064 nm, 30 mW·mm⁻² collimated laser beam showed different temperature gradients (FIG. 17). Statistical analysis (Student's T-test) was conducted between results from each set of two consecutive GNRs concentrations to assess significance. It was observed that at 808 nm, the significance was higher (P<0.001) for concentrations ranging from 0.1 to 1 nM. The maximum temperature did not increase as dramatically from 1 to 3 nM concentrations (P<0.01). This may be attributable to the saturation of photon absorbance by GNRs at higher concentration for a fixed power density. In contrast, at 1064 nm, GNR temperature did not increase as much as the 808 nm wavelength, though the control test with distilled water demonstrated a significant increase in ΔT (4.8° C.). This could be attributed to the higher light absorption rate of water at 1064 nm range. The thermal camera captured temperatures at different spots of the cuvette filled with GNRs. During the experiment, it was observed that maximum heat was generated at a 12 mm depth from the top surface of GNR solution in the cuvette. GNRs absorbed more light and gradually increased the temperature within this top layer as the concentration increased. Most of the light scattering and absorption occurred within the upper layer (˜20 mm) of GNRs while the bottom of the cuvette showed very little temperature increase (1-2° C.). Low concentration (0.1 nM) GNR solution raised the temperature (ΔT) by 5° C., which increased almost 3-fold for the 1 nM solution. The rate of temperature rise was directly proportional to the GNR solution concentration. The 3 nM solution rapidly increased the temperature by 10° C. within 1 min, a rate which may be too fast to control during the application of PPTT. Choosing the correct GNR concentration and laser exposure will be critical for generating sufficient heat to cause tissue hyperthermia while minimizing damage to nearby healthy tissues. This experiment demonstrated the light-to-heat conversion efficiency of different GNRs concentrations at 808 nm, the targeted wavelength for PPTT application in this study.

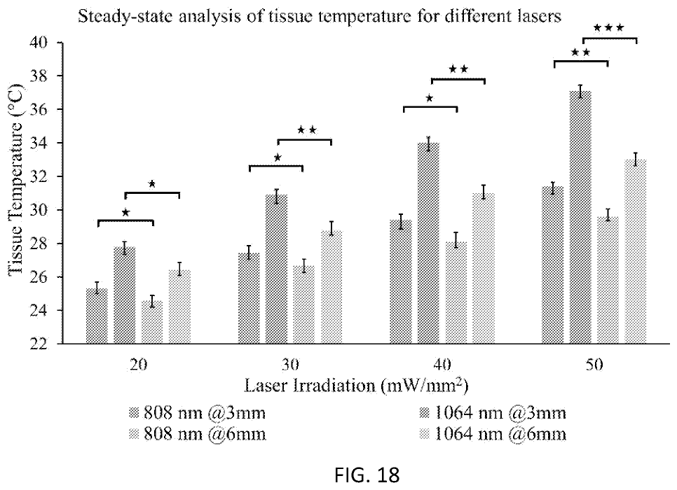

Tissue Photothermal Heating by Collimated Laser Beam. In this experiment, ex vivo tissue samples were photothermally heated by 808 and 1064 nm laser irradiation within the 20-50 mW·mm⁻² range with a 50% increment rate. Thermocouples inserted into the tissue detected temperatures at 3 and 6 mm depth from the top surface. Maximum steady state temperature and time were recorded for each laser irradiance. Results the separate sets of experiments were compiled together in a single plot to facilitate comparison of tissue photothermal heating at a fixed depth and laser irradiation for both wavelengths (FIG. 18). FIG. 18 represents the mean value of five experimental repetitions with an error bar representing one standard deviation (1σ).

Analysis of the results revealed that, tissue temperature increase (ΔT=difference between the tissue initial and final temperature) at 808 nm was lower than 1064 nm wavelength for similar irradiations: 51.4=5.5% and 65.8±3.2% lower at 3 mm and 6 mm tissue depth, respectively. ΔT increased linearly with respect to laser power for both wavelengths. Maximum ΔT was 15.1° C. for 1064 nm, 50 mW·mm⁻² irradiances at 3 mm tissue depth. No visible tissue damage was observed during the experiment. For similar laser irradiations, the rate of temperature increase (ΔT/time for tissue heating) was 95.6±4.8% and 87.7±6.1% higher for 1064 nm relative to 808 nm at 3 and 6 mm tissue depths, respectively. This finding is in good agreement with literature reports and prior work from our group on porcine pancreas tissue optical properties measurements [14,15,41]. It is evident from FIG. 18 that low irradiation by 808 nm, i.e., 20/30 mW·mm⁻² would not increase the tissue temperature as much as 1064 nm. It also implies that if the laser exposure time is reduced, the 808 nm wavelength would be less harmful to the healthy surrounding tissue than the 1064 nm. Hence, the 808 nm will be more selective for photothermal ablation of pancreas tissue than the 1064 nm when paired with functionalized GNRs of the appropriate resonance to specifically target the tumor region only. The 1064 nm wavelength may be helpful for approaches wherein deep penetration and rapid temperature rise are desirable.

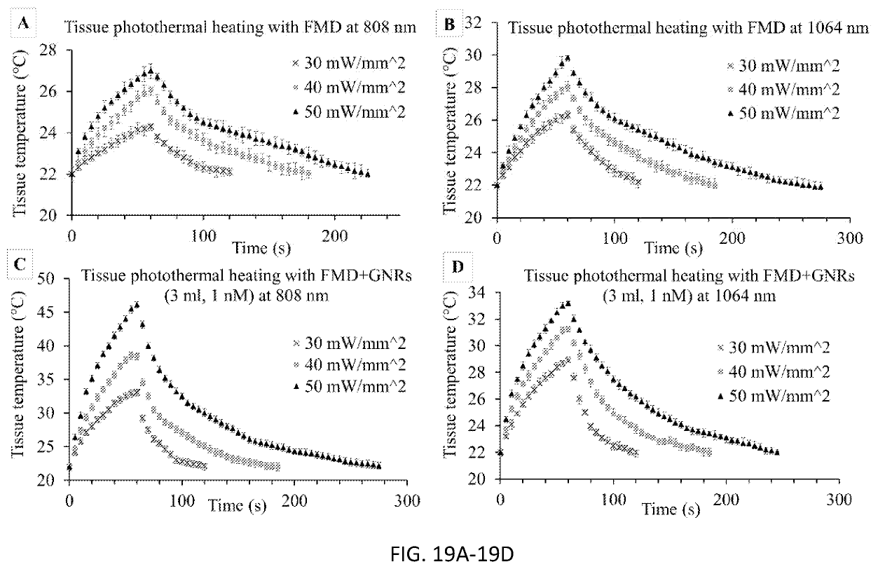

Tissue Photothermal Heating by FMD) and with without GNRs. The purpose of this set of experiments was to evaluate the FMD's effectiveness in photothermal heating of ex vivo porcine pancreas tissue with and without local GNR solution. The small size of the FMD tip (silica core area=0.25 mm²) focused the high-energy laser to the specific tissue area, which resulted in rapid and concentrated heating. Concentrated irradiation caused tissue burning and carbonization at the FMD tip when the applied laser intensity was >60 mW·mm⁻² or the exposure time was too long. Preliminary tests established the range of laser intensities (30-50 mW·mm⁻²) and exposure times (60 s) for both 808 and 1064 nm wavelengths to avoid tissue burning at the FMD tip. The results of these experiments are illustrated in FIG. 19A-19D). The first set of experiments (FIGS. 19A and 19B) evaluated FMD irradiation without GNRs while the next set (FIGS. 19C and 19D) explored irradiation with locally infused GNRs.

Initial irradiation experiments without GNRs exhibited faster rates of temperature increase (ΔT/time) for 1064 nm wavelengths than the 808 nm at different intensities. For 808 nm, ΔT after 1 min was measured at 2.3±0.6, 4.1±0.4, and 5.1±0.5° C. for 30, 40, and 50 mW·mm⁻² irradiances, respectively. For similar irradiances at 1064 nm, ΔT was almost double (4.3±0.4, 6.1±0.7, and 8.2±0.6° C.). These results followed a similar trend as observed in previous experiment with collimated beam photothermal heating for similar laser irradiations. The difference between the photothermal heating through the collimated beam and FMD are the photons distribution and the exposed tissue area. The collimated beam uniformly distributes the photons over a larger area compared to the FMD tip where photons spread out in a much smaller tissue area resulting in a rapid temperature increase. During the application of the PPTT, this phenomenon will help in selectively heating a tissue volume of interest.

The next set of experiments included local infusion of GNR solution prior to irradiation with the same wavelengths and irradiances as the prior set of experiments. The GNR concentrations were increased (0.1, 0.25, 0.5, 0.75, and 1 nM) while keeping the total infused volume constant (1 ml). FIGS. 19C and 19D exhibit the results for 1 nM GNR solution, as it provides a good representation of the observed trends. At 1064 nm, ΔT increased by 2.6±0.4, 3.1±0.5, and 3.6±0.3° C. due to the inclusion of GNRs at 30, 40, and 50 mW·mm⁻², respectively. In contrast, at 808 nm, ΔT increased significantly by 8.8±0.5, 12.5±0.5, and 19.1±0.6° C. while comparing the results between FIGS. 19A and 19C for similar irradiances. Following 808 nm exposure to pancreatic tissue with infused GNRs, visible bleaching around the FMD insertion area was evident. However, no tissue carbonization was observed during the 60 s irradiations for any of the experiments.

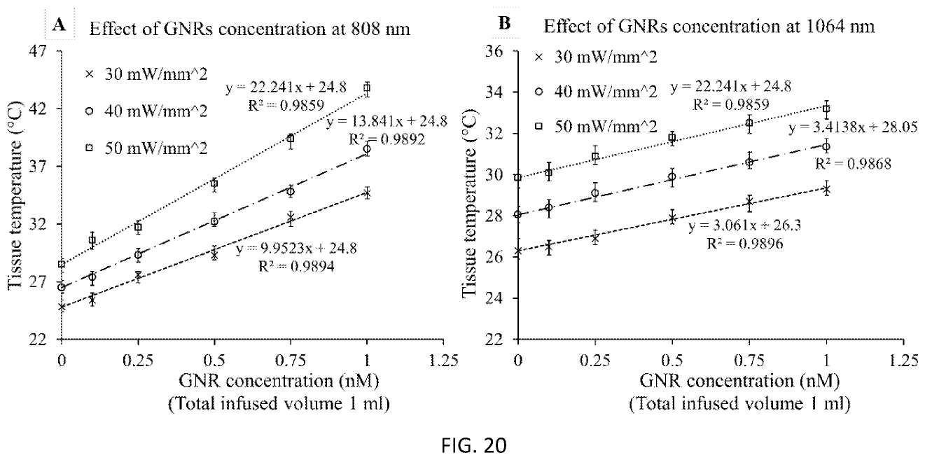

FIG. 20 depicts the effect of GNR concentration on ex vivo tissue photothermal heating (t=60 s) at 808 and 1064 nm for three laser irradiances (30, 40, and 50 mW·mm⁻²). A linear trend was observed between tissue temperature rise and GNR concentration (R²=0.98). Interestingly, the gradients at 808 nm were approximately 3-5 times higher than 1064 nm (For example, at 40 mW mm⁻² irradiation, the slopes were 12.1±0.5° C./nM and 3.2±0.3° C./nM at 808 and 1064 nm, respectively). Slopes of the trendlines for 1064 nm were nearly identical whereas they demonstrated a slightly increasing trend for 808 nm. The steeper slope at 808 nm indicates a higher degree of temperature increase for a small increment of GNRs concentration. It is evident that the 1064 nm temperature increased overreached the 808 only when no GNRs were present, but that GNR infusion had a far more significant effect on the 808 nm irradiation. It is also worth noting that, within the laser irradiances and GNR concentrations studied, there was no visible plateauing of the heating obtained. This indicates that these experiments did not reach a saturation point, or a concentration wherein nearly 100% of the light was absorbed and that further increases would not lead to increases in absorption.

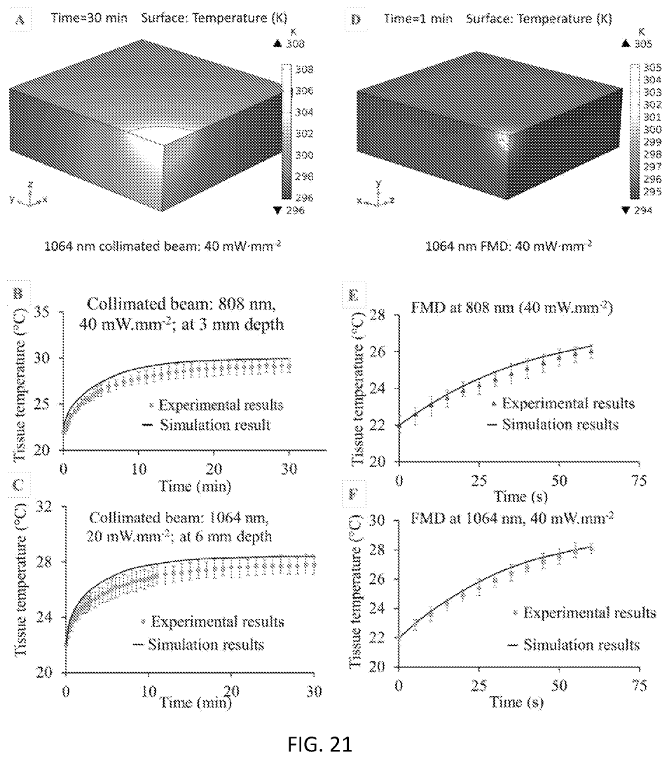

Computational Modelling. A computational model was developed in COMSOL Multiphysics® based on the Pennes bioheat equation and the time dependent light diffusion approximation. The model utilized tissue-specific thermal and optical parameters for the precise prediction of photon distribution and photothermal heating of pancreatic tissue. The model was modified to study the effects of two different heat sources: a collimated laser beam and light emission from the FMD tip. Tissue temperatures for 808 and 1064 nm laser wavelengths, as well as different laser irradiances, were recorded and compared against experimental results using similar conditions. Some key findings were illustrated in FIG. 21 where the left-hand column (FIG. 21A, 21B, 21C) represents results from collimated beam exposure, and the right-hand column (FIG. 21D, 21E, 21F) represents results from tissue heating through the FMD tip.

In the experiments using the collimated beam, tissue temperature gradually increased until it reached a steady state (temperature change <0.01° C. for 5 min). The temperature readings obtained from the experiments were plotted alongside the simulation results. Though the initial tissue temperatures for simulation were in close agreement with the experiments, they gradually deviated with respect to time and depth into the tissue. The difference between theoretical and experimental data was calculated as the average of the deviation between data sets. The average deviations between both data sets were 1.2±0.4° C. and 1.7±0.5° C. for 808 and 1064 nm, respectively. The computational model over-predicted the temperature by 3.3±0.6% and 3.7±0.5% for 808 and 1064 nm, respectively. In case of FMD, it was observed from the simulation that maximum heat was generated at the core of the fiber and the edges. The comparison between simulation and experiment for FMD tip followed the same procedure as the collimated beam model. The average deviations between simulation and experimental values were 4.1±0.8% and 3.8±0.7% for 808 and 1064 nm, respectively.

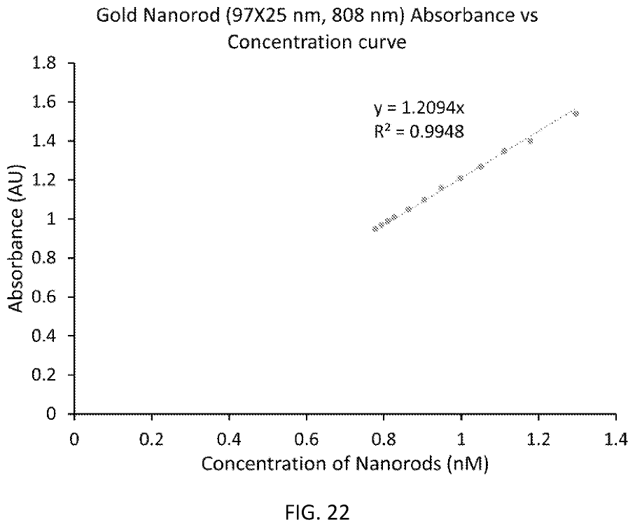

FIG. 22 was constructed by measuring the optical absorbance (at 808 nm) of a number of known concentrations of GNRs solutions through UV-Vis spectrometer. A trendline was plotted through the origin which reflects the linear relationship between optical absorbance and GNRs concentrations.

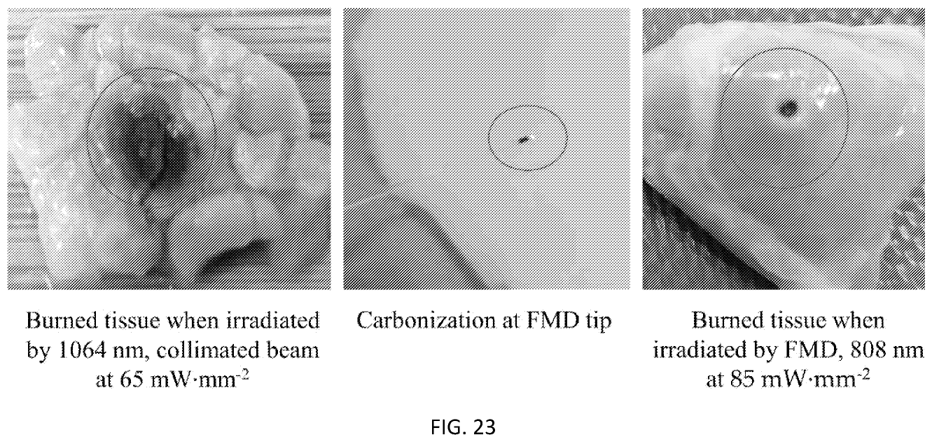

FIG. 23 shows the preliminary test results to find the laser irradiation range for ex vivo porcine pancreas heating. In this test, collimated laser irradiation (both 808 and 1064 nm) was gradually increased from 10 mW·mm⁻² with an increment of 5 mW mm⁻² in every 60 s. Tissue temperature was monitored using a thermal camera. At 1064 nm wavelength, when the irradiation was set at 65 mW mm⁻², the tissue started to burn with visible smoke coming for the tissue surface and a rapid temperature rise to more than 100° C. A similar condition was observed for 808 nm wavelength, when the irradiation was set to 85 mW·mm⁻². After tests, tissue samples were preserved in formalin buffer solution for further investigation. Tissue samples were sectioned with scalpel to investigate the penetration depth of the burning. It was observed that tissue burning penetrated >5 mm into the tissue thickness. FMD laser irradiation showed a similar result with carbonization at the FMD tip due to the proximity to the burned tissue. This carbonization at FMD tip and on the tissue surface absorbed photon energy at a much higher rate, resulting in faster temperature rise, and propagation of burning. It can be noted that, FMD can withstand a much higher laser irradiation (20 W·mm⁻²). To avoid unwanted tissue burning, laser irradiation range was set up to 50 mW·mm⁻² for the current study.

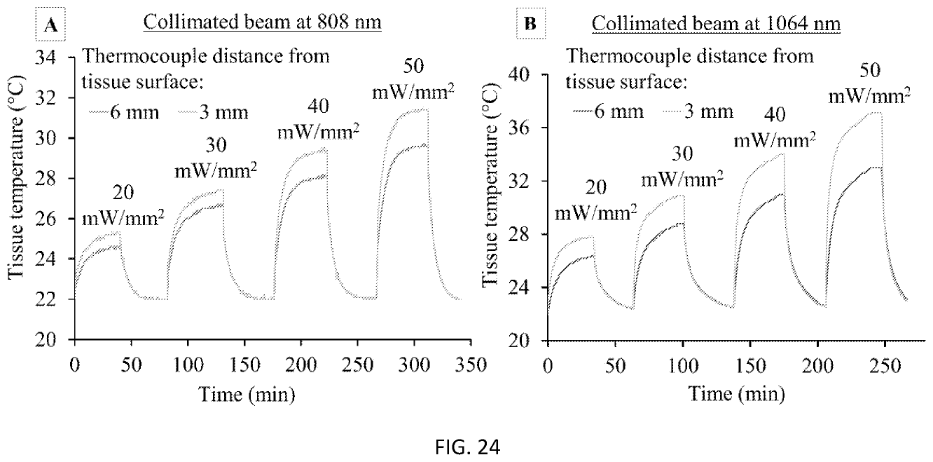

FIG. 24 shows the tissue temperature change with time when exposed to the collimated laser beam (808 and 1064 nm). Tissue sample was heated until the temperature reached a steady state. Then the laser was turned off to let the tissue cool down at room temperature. The sample was replaced after each single heating and cooling cycle. Same experiment was repeated with a different laser irradiation. Separate experiments were connected together to show a continuous graph of sequential heating and cooling cycles.

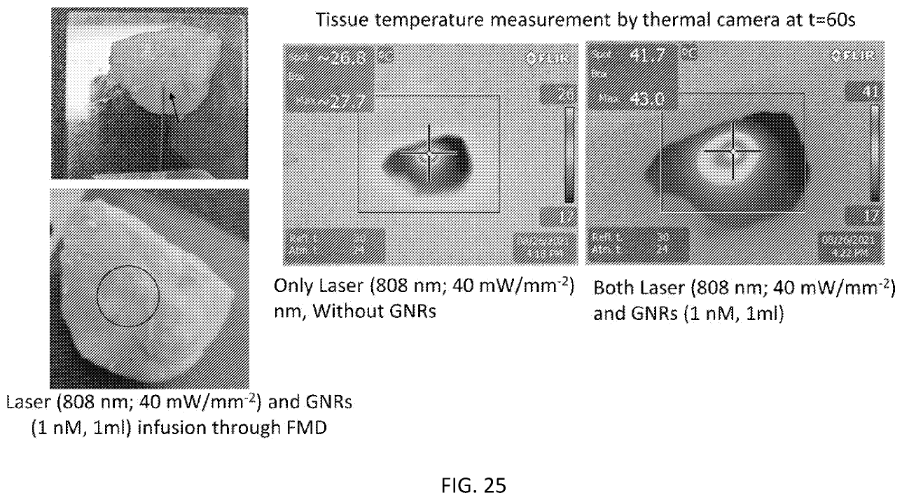

FIG. 25 shows the tissue temperature measurement technique through thermal camera imaging while transferring the light and GNRs solution by FMD.

The deviations between the simulation results and experimental values could be attributed to the optical loss due to the change in tissue physiological properties. Prior work on porcine pancreas optical properties revealed that the light transmittance and reflectance values change according to the tissue condition (fresh vs frozen samples). Increased diffuse reflectance of frozen samples might cause less photon fluence and absorption in these experiments. These deviations, however, were relatively small as the computational model utilized tissue-specific properties for fresh/frozen samples from Akhter et al., to mimic the experimental conditions. Also, the computational model assumed all the photons from the light source were incident on the tissue surface and propagated through the tissue. In reality, optical loss can occur during photon propagation from one medium to another due to reflection and back-scattering. Moreover, the literature review revealed a gap in Cₚ value (specific heat at constant pressure) for porcine pancreas tissue. The specific heat for porcine pancreas tissue at constant volume (Cᵥ) was reported in recent literature and used in this study with the assumption that, at low temperatures (22° C.), the difference between Cₚ and Cᵥ was negligible. To assess the sensitivity of the model to this parameter, additional simulations were conducted using the Cₚ value reported for human pancreas by Agafonkina et al., These simulation results showed a higher deviation (>5%) when compared to the experimental results of porcine pancreas tissue. This indicates that using appropriate tissue-specific properties has a considerable impact in developing accurate computational models.

Experimental results showed that plasmonic heating of GNRs at 808 nm resulted in rapid temperature increase due to quick light absorption and heat dissipation. Therefore, the 808 nm laser could be beneficial for PPTT, as the GNRs would absorb most of the applied laser light and cause hyperthermia to the specific tissue location while minimizing thermal damage to the healthy surrounding tissue due to the laser exposure. The effects of hyperthermia in in vivo tissue usually start at ΔT≥5° C. The current study showed that, for ex vivo porcine pancreas tissue, ΔT=5° C. was achieved within 60 s through plasmonic heating of GNRs (0.75-1 nM) with a laser irradiance of 30 mW·mm⁻² at 808 nm. Without GNRs and with an identical laser intensity, heating of only 2° C. ΔT over 60 s was measured, indicating selective hyperthermia of the surrounding tissue can be avoided.

Laser irradiation can be minimized by leveraging the linear relationship between tissue temperature rise and GNRs concentration as shown in the current study. Higher GNR concentrations could be harmful following therapy as they tend to accumulate in the liver, spleen, and kidney and remain for a time that varies with nanoparticle size and geometry. To achieve a minimum concentration, bio-conjugation of antibodies or other targeting moieties to the surface of the GNRs to selectively localize within the target region and/or individual cells has been previously demonstrated in the literature. Use of the FMD will also allow direct infusion within a tissue volume of interest, further reducing the amount of GNRs necessary to achieve therapeutic effect. As the dense stroma surrounding PDAC tends to provide a barrier to local molecular transport, the 1064 nm wavelength can be used to slightly heat the targeted tissue volume and increase local diffusive, convective, and bulk transport.

There were some limitations involved in the ex vivo tissue photothermal experiments. One of the limitations was using frozen tissue samples instead of fresh ones in some of the experiments which might affect the light absorption and photothermal heating process. Prior research revealed how thermal and optical properties change between fresh and frozen porcine pancreas tissue. The current study prioritized using fresh tissue samples, but frozen samples (up to 14 days) were also included due to sourcing issues. Faster degradation of porcine pancreas tissue could affect the steady state photothermal heating experiments. To minimize error, tissue samples were replaced after each single heating and cooling cycle. Also, tissue samples were hydrated periodically (every 5 min) by 4-5 droplets of phosphate buffer saline (PBS) to minimize the effect of tissue dehydration during long experiments. Other limitations may be attributed to the temperature measurement techniques utilized, as they may involve error due to the resolution of the thermal camera, sensitivity, and tolerances. In addition, differences between the ex vivo conditions studied, and the in vivo case should be noted. All studies were conducted at room temperature (22° C.) rather than normothermia for human tissue (˜37° C.), which might affect the tissue temperature rise relative to irradiation. This is anticipated, as pancreas tissue thermal conductivity and specific heat properties are dependent on tissue temperature. Moreover, as in vivo tissue has the heat sinking effects of vascular response and blood perfusion, as well as the heat generation/retention effects of metabolic activity and thermal insulation by surrounding organs, this study will need to be extended into in vivo models to properly capture the more complex response.

Precise control of different parameters of PPTT are needed for clinical implementation. These parameters include GNR size and concentration, laser wavelength and intensity, and the tissue thermal and optical properties. While it is necessary to start the characterization process with ex vivo experiments, the limitations for optimizing therapeutic dosage must be understood. The computational model presented will be used to further optimize the parameters and approach for this therapeutic modality. An FEM model has been presented to better understand and predict the porcine pancreas photothermal heating with 808 and 1064 nm laser wavelengths. This model accurately predicted experimental tissue temperature profiles (within a 3-5% error margin) via implementation of tissue-specific thermal and optical property measurements. The plasmonic photothermal heating model of the GNRs developed earlier by our group can be coupled to this current model for predicting the PPTT in the in vivo environment. This extension of the computational model as well as the in vivo application would be the focus of future research that would facilitate the implementation of PPTT for the treatment of pancreatic cancer.

### B. Material and Methods

Tissue Sample Collection. Porcine pancreas tissue samples were obtained from a USDA-approved abattoir. Immediately after the animals were sacrificed (<10 minutes), the pancreas was excised, and placed in separate Ziploc bags. An insulated cooler box with ice was used for transporting the samples to the laboratory. Upon arrival to the laboratory, the tissue samples were washed in phosphate-buffered saline (PBS) solution. Samples were sliced in a 3×3 cm² cross-section area using a scalpel and the dimensions were estimated using calipers and image processing software (ImageJ). Samples used in different experiments had a thickness of approximately 1±0.05 cm. A portion of these tissue specimens was utilized in running experiments on the same day of collection (referred to as ‘fresh’ tissue samples). The rest of the specimens were stored individually in a −62° C. freezer for later use within 7-14 days (referred to as ‘frozen’ tissue samples). All study procedures were completed following approved protocols by the University of Texas at San Antonio's Institutional Biosafety Committee.

GNRs Synthesis and Photothermal Heating. GNRs were synthesized by a seed-mediated growth protocol described in the literature. Detailed method of the synthesis process was described previously by this group (Manrique-Bedoya et al., The Journal of Physical Chemistry C 2020, 124, 17172-82). The optical absorbance of the nanorods was confirmed via UV-Vis spectroscopy (400-1100 nm) and their geometry (length, width, aspect ratio) was measured via SEM (scanning electron microscopy) imaging and analyzed in ImageJ. As-synthesized GNRs (i.e. non-functionalized) were utilized in this experiment where GNRs were suspended in CTAB solution (Cetyltrimethylammonium bromide). The initial concentration of the solution was 3.2 nM, which was evaluated from the mass percentage of gold in a known volume of GNR solution (3.5 ml cuvette). The mass percentage was measured by quantifying the weight of gold through centrifuging the solution and subtracting the weight of water. This concentrated solution was serially diluted by adding 10 ml of distilled water in each increment. The optical absorbance of each diluted solution was obtained from the UV-Vis spectrometer. An absorbance vs GNR concentration graph was plotted from these optical measurements which was the basis for identifying any unknown GNR concentration from the optical absorbance measurement.

Next, the maximum steady state temperature of different GNR concentrations (0.1, 0.25, 0.5, 0.75, 1, 3 nM) were measured at fixed laser irradiations (30 mW·mm⁻² at 808 and nm). The experimental setup includes a transparent cuvette (3.5 ml) filled with a known concentration of GNR solution exposed under the collimated laser beam. The distance between the laser pointer lens and the GNR top surface was fixed at 3 cm. The cuvette cross-sectional area (100 mm²) was significantly larger than the collimated beam area (24 mm²), which ensured the unobstructed interaction between the laser and GNRs. A thermal camera (E5, FLIR, Wilsonville, Oregon) was positioned 30 cm away to capture a lateral image of the cuvette and laser. The camera setting was adjusted to measure temperature at three different spots on the cuvette: 2 mm, 12 mm, and 20 mm below the top surface of the GNR solution. The purpose was to observe the temperature distribution as a function of depth into the GNR solution and evaluate the mean value of these three measurements. The thermal camera was connected to a computer for real-time data collection at 5 Hz. Laser irradiation continued until the GNR temperature reached a steady state (temperature change <0.05° C. for 5 min). The experiment was repeated 5 times for each concentration of the solution (n=5). A control test was conducted following similar procedure with distilled water (blank) for comparison.

Tissue Photothermal Heating with Collimated Laser Beam. The photothermal heating experiments utilized two different continuous wave laser sources with collimated beam outputs at 808 nm (LRD-0808-PFR-01000-03, Laserglow Technologies) and 1064 nm (YLR 10-1064-LP, IPG photonics) wavelengths. The collimated beam areas for both laser sources were estimated by taking a thermal image (E40, FLIR thermal camera) of the beam reflection and post-processing the image using ImageJ. The beam areas were measured at 24 and 19.6 mm² for the 808 and 1064 nm, respectively. Collimated beam output power was measured by an integrating sphere (photo detector, 819D-UV-2-CAL, Newport, Franklin, MA) and an optical power meter (1936-R, Newport, Franklin, MA). The beam areas were used to set the beam intensity within the range of 20-50 mW·mm⁻² (in 10 mW·mm⁻² increments) for both laser sources. This range was selected through a set of preliminary tests with different laser irradiations which demonstrated that >60 mW·mm⁻² irradiation would cause unwanted tissue burning. The tissue specimen was placed on a glass slide (12×12×0.5 cm) beneath the laser attached to an adjustable holder. The distance between the tissue top surface and the laser was kept constant at 4 cm. Two K-type thermocouples (Fluke Co; Everett, WA) were inserted into the tissue at 3 and 6 mm below the tissue surface while ensuring they were 1-2 mm from the collimated beam path. An Omega thermometer (OM-HL-EH-TC, Omega Engineering, Norwalk, CT) was utilized to record the temperature reading from the thermocouples at a set frequency of 5 Hz. Both thermocouples were calibrated against a cold junction (ice) before conducting the experiments. A glass thermometer was used to measure the room temperature. The experiment started when the tissue initial temperature reached room temperature (22° C.). Laser emission continued until tissue temperature reached a steady state maximum range, i.e., temperature fluctuation was <0.05° C. for 5 min. At this point, the laser was turned off to allow the tissue specimen to cool normally (no forced convection) to room temperature. The experiment was repeated 5 times with different specimens (n=5). A similar procedure was followed for experiments with two different laser sources and four different light intensities.

Tissue Photothermal Heating with FMD) and with without the GNRs. The fabrication process, optical performance, and mechanical characterization of the FMD were described previously (Akhter et al., Journal of the Mechanical Behavior of Biomedical Materials 2020, 112, 104042). In brief, the FMD utilized a flexible light guiding capillary (fused silica, 365 μm outer diameter, and 150 μm inner diameter) which can co-deliver light (both visible and NIR) through the annular silica core and liquid through the hollow bore (FIG. 14). The tip of the FMD can be either flat polished or beveled at a specific angle for easy penetration of soft tissue. The device was designed for connecting to a laser source and a syringe for co-delivering light and fluid, respectively. The goal of using FMD in this study was to assess its utility as a delivery vehicle for GNRs and thereby PPTT. The cross-section area of the annular silica core of a flat polished FMD tip was evaluated from capillary geometry (0.086 mm²) to identify the applied laser intensity. FMD tips were inserted horizontally (2-3 mm) into the tissue top surface lying on a glass slide at room temperature (22° C.). The laser exposure time was of 60 s was decided upon through discussion with clinical collaborators at University of Texas Health Science Center at San Antonio. The aim was to complete the procedure in a short time to avoid unwanted heating of healthy surrounding tissue. A set of preliminary tests were conducted to evaluate the threshold light intensity from the FMD tip that would induce tissue carbonization, which would be an unwanted outcome to avoid. It was observed from preliminary tests that laser intensities >60 mW·mm⁻² from the FMD tip for both 808 and 1064 nm wavelengths could cause tissue carbonization within the 60 s exposure period. Hence, FMD tip output laser intensity was set sequentially at 30, 40, and 50 mW mm⁻² for this test. A thermal camera was positioned above the tissue sample at a fixed height of 30 cm to capture the tissue temperature profile. The camera was connected to a computer for recording temperature data at a 2 Hz sampling rate. After 60 s, the laser was turned off, and the tissue was allowed to cool down to room temperature. The test was repeated five times with different tissue samples for each light intensity at both 808 and 1064 nm (n=5).

GNR infusion and tissue photothermal heating through FMI). Another set of experiments were conducted to assess the combined effect of laser irradiation and GNR concentration on ex vivo porcine pancreas tissue photothermal heating. The experimental setup was similar to the previous experiments except for the addition of GNR solution transfused through the FMD by a syringe pump. The FMD was also coupled by free coupler to the 808 or 1064 nm laser, which was delivered at the same irradiances as before (30, 40, and 50 mW·mm⁻²). GNR concentrations infused included 0.1, 0.25, 0.5, 0.75 and 1 nM delivered at 1 mL/min for 60 s. Laser irradiation initiated right after the infusion of GNRs at 60 s. Tissue temperature was monitored through the thermal camera following the same instruction as mentioned earlier. Laser irradiation continued for 60 s followed by the natural convective cooling to room temperature (22° C.). The experiment was repeated five times for each laser irradiation and GNR concentration (n=5). The tissue specimen was replaced between each experiment.

Computational Modelling of Laser-Tissue Interaction. A computational model was developed to predict the tissue temperature rise induced by photothermal heating at the 808 nm and 1064 nm wavelengths. The modelling procedure was adapted from Feng et al. (Engineering with computers 2009, 25, 3-13) and Saccomandi et al. (IEEE Transactions on biomedical engineering 2012, 59, 2958-2964) with the inclusion of the tissue specific thermal and optical properties at both laser wavelengths from our previous work. The 3D model was developed using the FEA software COMSOL Multiphysics® with a 3×3×1 cm³ block representing the tissue sample and collimated laser beam simulated by a circular area at the center of the block. Due to axial symmetry, only a quarter of the model is required to perform the simulations.

The effect of photothermal heating induced by the laser beam can be modeled by the Pennes bioheat transfer equation. Assuming the model is a representation of laser heating in ex vivo tissue, the blood perfusion and metabolism contributions can be neglected. Thus, the bioheat equation reads:

$${ρ C}_{p} \frac{\partial T ( x , t )}{\partial t} - ∇ . ( k ∇ T ( x , t ) = {Q}_{light} ( x , T ) \qquad ( 1 )$$

Here, ρ (kg·m⁻³), Cₚ (J·kg⁻¹ K⁻¹), k (Wm⁻¹ K⁻¹) are the tissue density, specific heat, and thermal conductivity, respectively. For simplicity, tissue was considered homogeneous and isotropic. T(x, t) is the tissue temperature, expressed as a function of space and time. Q_{light}(x, T) (W·m⁻³) is the heat source term due to photon absorption caused by the laser-tissue interaction, which can be expressed as follows:

$${Q}_{light} ( x , T ) = {μ}_{a} . Φ ( x , t ) . E \qquad ( 2 )$$

Here, μₐ (mm⁻¹) is the absorption coefficient of tissue at a specific laser wavelength, Φ(x, T) (m⁻²·s⁻¹) is the photon fluence (number of photons passing through a unit area at a point in space per unit time), and E (J) is the photon energy. The photon energy can be evaluated as

$$E = \frac{h c}{λ} \qquad ( 3 )$$

with h (6.63×10⁻³⁴ J·s) being Plank's constant, c (2.99×108 m·s⁻¹) the speed of light, and λ (m) the laser wavelength. Additionally, the photon fluence in the tissue (i.e. Φ(x, t) in Equation 2) can be estimated from the time dependent light diffusion approximation derived from the radiative transfer equation [48]. With no additional sources apart from light absorption, the equation reads:

$$\frac{1}{c} \frac{\partial }{\partial 𝔱} Φ ( x , t ) + ∇ . ( - D ∇ Φ ( x , t ) ) = - {μ}_{a} . Φ ( x , t ) \qquad ( 4 )$$

Here, μₐ (m⁻¹) is the absorption coefficient of tissue at a specific light wavelength, and D is the optical diffusion coefficient which depends on tissue-specific optical properties. The diffusion coefficient can be expressed as [49,50]:

$$D = \frac{1}{[ 3 ( {μ}_{a} + {μ}_{s}^{′} ) ]} \qquad ( 5 )$$

Where μₛ′(m⁻¹) is the reduced scattering coefficient of tissue at a specific laser wavelength.

Equations 1 and 4 were solved using the heat transfer and general form PDE modules in COMSOL Multiphysics®, respectively. Tissue initial temperature (T_{|t=0}) was set at 22° C. to mimic the ex vivo experiment. The collimated laser beam on the tissue surface was modeled using a Dirichlet boundary condition:

$$Φ {\vert}_{source} = ( 1 - R ) \frac{{w}_{0}}{π {r}_{0}^{2} ( 1 - E )} \qquad ( 6 )$$

With R being the light reflectance at the air-tissue interface for a specific laser wavelength, w₀ (W) the laser power, and r₀ (m) the collimated beam radius. In the current model, the laser power was assumed to be uniform over the source boundary. Thermal insulation boundary conditions (Neumann boundary condition) were imposed at the symmetry planes (left and front boundaries). Lastly, free convective cooling (q_{conv}) was imposed at the top, right, and back boundary area of the tissue.

$${q}_{c o n v} = {h}_{c o n v} ( T ( x , t ) - {T}_{∞} ) \qquad ( 7 )$$

Here, h_{conv} (5 W·m⁻²·K⁻¹) is the convection heat transfer coefficient, and T_{∞}(295.15 K) is the ambient temperature. Furthermore, porcine pancreas tissue properties were obtained from both literature (e.g. ρ=1040 kg·m⁻³ and Cₚ=3630 J·kg⁻¹ K⁻¹) [40,52] and our team's previous work on characterization of tissue thermal and optical properties (e.g. k=0.45 Wm⁻¹ K⁻¹, μₐ, μₛ′, and R (detail optical properties are shown in Table S1 of supplementary materials) [41]. After setting up the equations and boundary conditions, two temperature probes were placed at 3 and 6 mm below the tissue top surface and 2 mm away from the beam path to mimic the experimental setup described in section 2.2 (FIG. 4). The model was run to assess the steady state tissue temperature for a 0-60 min period when exposed to different laser irradiations (20, 30, 40, 50 mW·mm⁻²) at both 808 and 1064 nm wavelengths.

To assess the effect of photothermal heating using the FMD, the collimated beam area of the model was replaced by the annular core of the FMD. It was assumed that photons are uniformly distributed throughout the core area of the FMD and scattered at the tip. A quarter of the FMD tip was modeled using the same procedure described earlier for the collimated beam. Uniform laser power over the source boundary was assumed. The rest of the boundary conditions remained the same. The model was run for 60 seconds using 30, 40, and 50 mW·mm⁻² laser irradiations at both 808 and 1064 nm.

### REFERENCES

1. Rahib et al., 2014 Cancer research, 74 (11), pp. 2913-2921.
2. Smith et al., 2009 Journal of clinical oncology, 27 (17), pp. 2758-65.
3. Stocken et al., 2005, British journal of cancer, 92 (8), pp. 1372-81.
4. Strobel et al., 2019 Nature reviews Clinical oncology, 16 (1), pp. 11-26.
5. Lee and Lee, 2014 World journal of gastroenterology: WJG, 20 (24), p. 7864.
6. Di Matteo et al., 2018 Gastrointestinal endoscopy, 88 (1), pp. 168-74.
7. Jacques and Prahl, 2004, Oregon Graduate Institute.
8. Partovi et al., 1987, Lasers in surgery and medicine, 7 (2), pp. 141-54.
9. Huang et al., 2008, Lasers in medical science, 23 (3), p. 217.
10. Hood et al., 2013, Lasers in surgery and medicine, 45 (7), pp. 418-26.
11. Sultan, 1990, Lasers in Medical Science, 5 (2), pp. 185-193.
12. Huang et al., 2006, Journal of the American Chemical Society, 128 (6), pp. 2115-20.
13. Manrique-Bedoya et al., 2020, The Journal of Physical Chemistry C, 124 (31), 17172-182.
14. Bayazitoglu et al., 2013 International Journal of Heat and Mass Transfer, 67, 469-86.
15. Saccomandi et al., 2016 Lasers in medical science, 31 (6), 1041-50.
16. Saccomandi et al., 2012 IEEE Transactions on biomedical engineering, 59 (10), 2958-64.
17. Sazgarnia et al., 2015 Journal of thermal biology, 47, 32-41.
18. Valvano et al., 1985 International Journal of Thermophysics, 6 (3), 301-11.
19. Chandra et al., 2007 Journal of biomedical optics, 12 (6), 060501.
20. Bashkatov et al., 2011 Journal of Innovative Optical Health Sciences, 4 (01), 9-38.
21. Lee et al., 2013 Biomedical optics express, 4 (12), 2828-34.
22. Saccomandi et al., 2018, International Journal of Hyperthermia, 35 (1), 398-418.
23. Hu et al., 2017 Advanced Materials, 29 (33), 1700448.
24. Poudel et al., 2017 Colloids and Surfaces B: Biointerfaces, 160, 73-83.
25. Abramoff et al., 2004 Biophotonics international, 11 (7), 36-42.
26. URL formlabs-media.formlabs.com/datasheets/High_Temp_Technical.pdf.
27. Bergman et al., 2011, Fundamentals of heat and mass transfer, John Wiley & Sons.
28. URL dow.com/content/dam/dcc/documents/en-us/productdatasheet/11/11-31/11-3184-sylgard-184-elastomer.pdf?iframe=true, Mar. 20, 2020.
29. Sweat et al., 1973 Journal of Food Science, 38 (1), 158-60.
30. Patterson et al., 1989 Applied optics, 28 (12), 2331-36.
31. Prahl, International Society for Optics and Photonics, 1030509.
32. Prahl, 2011 Oregon Medical Laser Center, St. Vincent Hospital, 1344, 1-74.
33. Pickering et al., 1993 Applied optics, 32 (4), 399-410.
34. Prahl, 1999 Oregon Medical Laser Center, St. Vincent Hospital, 9205.
35. Chan et al., 1996 IEEE Journal of selected topics in quantum electronics, 2 (4), 943-50.
36. Oldenburg et al., International Society for Optics and Photonics, 50-60.
37. Cook et al., 2011 Biomedical optics express, 2 (11), 3193-3206.
38. Lai et al., 2010 Applied optics, 49 (16), 3235-38.
39. Khalifa, 2001 Energy conversion and management, 42 (4), 491-504.
40. Ritz et al., 2001 Lasers in Surgery and Medicine 29 (3), 205-12.
41. Shimizu et al., Pancreas, 5 (1), 91-94.
42. Sebeke et al., 2020 International Journal of Hyperthermia, 37 (1), 786-98.
43. Akhter et al., 2020 J. Mechanical Behavior of Biomedical Materials, 112, 104042.
44. Hood et al., 2015 Engineering, 1 (3), 344-50.
45. Hood et al., 2013 Lasers in surgery and medicine, 45 (3), 167-74.
46. Hood et al., 2013 Lasers in medical science, 28 (4), 1143-50.
47. Murphy and Marks, 1999 Journal of food process engineering, 22 (2), 129-40.
48. Pancreatic Cancer 2018 URL seer.cancer.gov/statfacts/html/pancreas.html.
49. Levy et al., 2017 Gastrointestinal endoscopy 86 (1): 161-9.
50. Nishimura et al., 2018 Gastrointestinal endoscopy 87 (4): 1126-31.
51. Yoo et al., 2016 Journal of gastrointestinal oncology 7 (6): 1019.
52. Tsen et al., 2017 Pancreas 46 (7): 839-49.
53. Gaidhane et al., 2012 Gastroenterology research and practice 2012.
54. Di Matteo et al., 2010 Gastrointestinal endoscopy 72 (2): 358-63.
55. Di Matteo et al., 2018 Gastrointestinal endoscopy 88 (1): 168-74
56. Kennedy et al., 2011 Small 7 (2): 169-83.
57. Hood et al., 2011 Journal of medical devices 5 (3): 031012.
58. Lopez et al., 2019 SPIE Nanoscience+Engineering 11082 Epub gr.
59. David et al., MEDICAL PHYSICS; 2019: WILEY 111 RIVER ST, HOBOKEN 07030-5774, NJ USA.
60. Gray et al., 2020 Physics in Medicine & Biology.
61. Mondal et al., 2016 Pancreas 45 (7): 929-33.
62. Dickerson et al., 2008 Cancer letters 269 (1): 57-66.
63. Nikoobakht et al., 2003 Chem Mater 15 (10): 1957-62.
64. Jana et al., 2001 J Phys Chem B 105 (19): 4065-7.
65. Jana et al., 2001 Adv Mater 13 (18): 1389-93.
66. Manson et al., 2011 Gold bulletin 44 (2): 99-105.
67. Troiani et al., 2012 Current drug targets 13 (6): 802-10.
68. Hermanson et al., 1996 Bioconjugate Techniques, 1st ed: Academic Press 785.
69. Peppas, 2019 Hydrogels in medicine and pharmacy: fundamentals: CRC press.
70. Chen et al., 2008 Magnetic resonance imaging. 26 (10): 1433-41.
71. Madsen et al., 1978 Medical physics 5 (5): 391-4.
72. Lindsey et al., 2017 IEEE transactions on ultrasonics, ferroelectrics, and frequency control 64 (7): 1078-86.
73. Fatin-Rouge et al., 2004 Biophysical journal 86 (5): 2710-9.
74. Pearce, editor. 2009 International Society for Optics and Photonics.
75. He et al., 2009 Journal of biomechanical engineering 131 (7).
76. Feng et al., 2008 Journal of biomechanical engineering 130 (4): 041016.
77. Hood et al., 2013 Lasers in medical science 28 (4): 1143-50.
78. Huang et al., 2008 Lasers Med Sci 23 (3): 217.
79. Hirsch et al., 2003 Proc Natl Acad Sci USA 100 (23): 13549-54.

1. A method of treating pancreatic cancer comprising:
(i) contacting a pancreatic cancer to be treated with a plasmonic photothermal agent forming a sensitized target;
(ii) exposing the sensitized target to light having a resonance wavelength of the plasmonic photothermal agent for a predetermined amount of time;
wherein the sensitized target is heated to a sub-hyperthermic temperature that reduces the size of the target.

2. The method of claim 1, wherein the wavelength of light is 500 nm to 1200 nm.

3. The method of claim 1, wherein the wavelength of light is 808 nm or 1064 nm.

4. The method of claim 1, wherein the wavelength of light is 808 nm.

5. The method of claim 1, wherein the wavelength of light is 1064 nm.

6. The method of claim 1, wherein the sensitized target is exposed to light for 1 to 30 minutes, one or more times.

7. The method of claim 1, wherein the sub-hyperthermic temperature is 38 to 42 C, preferably about 40 C.

8. The method of claim 1, wherein the plasmonic photothermal agent is a metallic nanoparticle.

9. The method of claim 8, where the metallic nanoparticles are gold nanorods.

10. The method of claim 9, wherein the metallic particles are coupled to an antibody having an affinity for the target.

11. The method of claim 1, wherein contacting further comprises guiding delivery of the plasmonic photothermal agent using an imaging system.

12. The method of claim 11, wherein the imaging system is an endoscopic ultrasound imaging system.

13. The method of claim 1, wherein contacting further comprises guiding delivery of the plasmonic photothermal agent and exposure of the plasmonic photothermal agent to light having a resonance wavelength of the plasmonic photothermal agent using a fiberoptic microneedle device (FMD).
